(19) United States
(12) Patent Application Publication
(10) Pub. No.: US 2023/0286918 A1
(43) Pub. Date: Sep. 14, 2023

(54) MANUFACTURING PROCESS FOR 3,5-DICHLOROPICOLINONITRILE FOR SYNTHESIS OF VADADUSTAT

(71) Applicant: Akebia Therapeutics, Inc., Cambridge, MA (US)

(72) Inventors: Valdas JURKAUSKAS, Cambridge, MA (US); Young Chun JUNG, Cambridge, MA (US); Taesoo KWON, Cambridge, MA (US); Arunachalam KANNAN, Cambridge, MA (US); Vijaya Bhasker GONDI, Cambridge, MA (US)

(73) Assignee: Akebia Therapeutics, Inc., Cambridge, MA (US)

(21) Appl. No.: 18/012,012

(22) PCT Filed: Jul. 1, 2021
(86) PCT No.: PCT/US2021/040138

§ 371 Date: Dec. 21, 2022

Related U.S. Application Data
(60) Provisional application No. 63/047,654, filed on Jul. 2, 2020.

(51) Int. Cl. C07D 213/81; C07D 213/84; C07D 401/04; B01J 31/02

(52) U.S. Cl. CPC C07D 213/81; B01J 31/0239; C07D 213/84; C07D 401/04; B01J 2231/005; B01J 2531/002

(57) ABSTRACT

Disclosed herein are methods and processes of preparing vadadustat or a pharmaceutically acceptable salts thereof, and intermediates (e.g., a compound of Formula (I), (I-F), (II), or (IV), or a pharmaceutically acceptable salts thereof) useful for the synthesis of vadadustat.

### CROSS-REFERENCE TO RELATED APPLICATIONS

The present application claims benefit of U.S. Provisional Application No. 63/047,654, filed Jul. 2, 2020, which is incorporated by reference in its entirety.

### BACKGROUND

Vadadustat ({[5-(3-chlorophenyl)-3-hydroxypyridine-2-carbonyl]amino}acetic acid) is a Hypoxia Inducible Factor Prolyl Hydroxylase inhibitor (HIF-PH inhibitor), which has emerged as a new drug that is highly useful for treating or preventing anemia such as, for example, anemia secondary to or associated with chronic kidney disease. While methods for synthesis of vadadustat have been described, there remains a need for new methods to manufacture vadadustat (including its intermediates) with improved overall yield and reduced costs.

### SUMMARY

The present invention is based, in part, on the surprising discovery that vadadustat or a pharmaceutically acceptable salt thereof, can be manufactured using the methods and compositions described herein. In particular, a compound according to Formula (I) (e.g., Compound 1), or Formula (II), or a pharmaceutically acceptable salt thereof — which can be used as an intermediate for manufacturing vadadustat — may be prepared using the methods and compositions described herein with unexpected efficiency.

Disclosed herein are methods and processes of preparing intermediates (e.g., a compound of Formula (I), (I-F), (II), or (IV)) or a salt thereof useful for the synthesis of vadadustat, and methods and processes for manufacturing vadadustat or a pharmaceutically acceptable salt thereof.

In one aspect, disclosed herein is a process for preparing a compound of formula (IV)

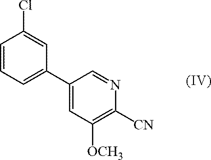 [SMILES: COc1cc(-c2cccc(Cl)c2)cnc1C#N]

or a pharmaceutically acceptable salt thereof, comprising:
(a) contacting a compound of Formula (I)

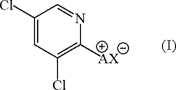

or a tautomer thereof, with a cyanide source to obtain a compound of Formula (II)

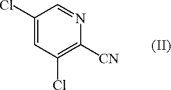

or a salt thereof, wherein A is an activator and X is a halogen; and
(b) contacting the compound of Formula (II) or a salt thereof with a compound of Formula (III)

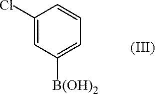

or a salt thereof, to form a compound of Formula (IVa),

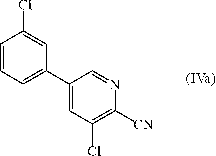 [SMILES: N#Cc1ncc(-c2cccc(Cl)c2)cc1Cl]

or a pharmaceutically acceptable salt thereof.

In another aspect, disclosed herein is a process for preparing a compound of formula (IV)

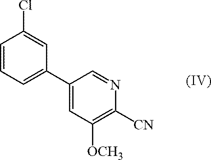 [SMILES: COc1cc(-c2cccc(Cl)c2)cnc1C#N]

or a pharmaceutically acceptable salt thereof, comprising:
(a-F) contacting a compound of Formula (I-F),

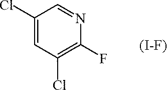

with a cyanide source to obtain a compound of Formula (II),

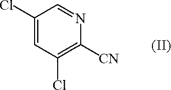

or a salt thereof; and
(b) contacting the compound of Formula (II) or a salt thereof with a compound of Formula (III)

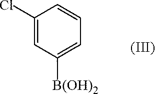

or a salt thereof, to form a compound of Formula (IVa),

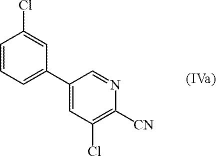 [SMILES: N#Cc1ncc(-c2cccc(Cl)c2)cc1Cl]

or a pharmaceutically acceptable salt thereof.

In embodiments, the process further comprising a step that is

(c) contacting the compound of Formula (IVa), or a salt thereof, with methanol or an inorganic salt thereof to form a compound of Formula (IV),

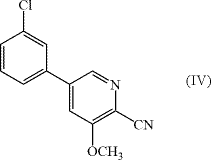 [SMILES: COc1cc(-c2cccc(Cl)c2)cnc1C#N]

or a pharmaceutically acceptable salt thereof.

In another aspect, disclosed herein is a process for preparing a compound of Formula (V)

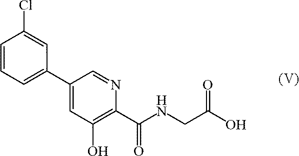 [SMILES: O=C(O)CNC(=O)c1ncc(-c2cccc(Cl)c2)cc1O]

or a pharmaceutically acceptable salt thereof comprising contacting a compound of Formula (IV) with glycine methyl ester hydrochloride, wherein the compound of Formula (IV) is obtained by the process described herein.

In embodiments, A is a nucleophilic amine, a triarylphosphine, or a trialkylphosphine.

In embodiments, A is DMAP (4-Dimethylaminopyridine), DABCO (1,4-diazabicyclo[2.2.2]octane), DBU (1,8-Diazabicyclo[5.4.0]undec-7-ene), DBN (1,5-Diazabicyclo[4.3.0]non-5-ene), triphenylphosphine, trimethylphosphine, or tributylphosphine.

In embodiments, A is DMAP.

In embodiments, X is F, Cl or Br

In embodiments, the compound of Formula (I) has the following structure,

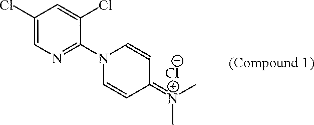 [SMILES: C[N+](C)=c1ccn(-c2ncc(Cl)cc2Cl)cc1 ; [Cl-]]

or a tautomer thereof.

In embodiments, the cyanide source is an inorganic cyanide salt or an organosilicon cyanide compound.

In embodiments, the cyanide source is NaCN, KCN, TMSCN (trimethylsilyl cyanide), Zn(CN)₂, or CuCN.

In embodiments, the cyanide source is NaCN.

In embodiments, step (a) or step (a-F) occurs in the presence of a solvent.

In embodiments, the solvent is an ester solvent, an aprotic solvent, or a combination thereof.

In embodiments, the ester solvent is isopropyl acetate (IPAc), ethyl propionate or ethyl acetate.

In embodiments, the aprotic solvent is methyl tert-butyl ether (MTBE), N-methyl-2-pyrrolidone (NMP), acetonitrile (ACN), acetone, heptane, methylene chloride (MC), tetrahydrofuran (THF), dimethylformamide (DMF), dimethyl sulfoxide (DMSO), or dimethyl acetamide (DMAc).

In embodiments, the aprotic solvent is tetrahydrofuran (THF), dimethylformamide (DMF), dimethyl sulfoxide (DMSO), or dimethyl acetamide (DMAc).

In embodiments, the aprotic solvent is methyl tert-butyl ether (MTBE), N-methyl-2-pyrrolidone (NMP), acetonitrile (ACN), acetone, heptane, or methylene chloride (MC).

In embodiments, step (a) occurs in the presence of a solvent that is IPAc.

In embodiments, step (a) occurs in the presence of a solvent that is a combination comprising IPAc and DMAc. In embodiments, IPAc and DMAc are present in a ratio ranging from about 19:1 to 1:1 IPAc:DMAc.

In embodiments, step (a) occurs at a temperature of about 80-100° C. or about 85-95° C.

In embodiments, step (a) occurs at a temperature of about 90° C.

In embodiments, step (a-F) occurs in the presence of a solvent that is NMP.

In embodiments, step (a-F) occurs in the presence of a solvent that is a combination comprising NMP and MTBE. In embodiments, NMP and MTBE are present in a ratio ranging from about 1:3 to 6:1 NMP:MTBE. In embodiments, NMP and MTBE are present in a ratio of about 3:2 NMP:MTBE.

In embodiments, a solvent (e.g., a solvent in step (a-F)) comprises water (e.g., comprises about 1% to 5% water). In embodiments, a solvent (e.g., a solvent in step (a-F)) comprises about 3% water.

In embodiments, step (a-F) occurs at a temperature of about 25-100° C.

In embodiments, step (a-F) occurs at a temperature of about 60° C.

In embodiments, the compound of Formula (I) is prepared by a process comprising: (i) contacting a compound of Formula (VI) or a salt thereof

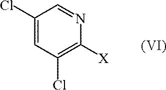

with the activator, wherein X is a halogen.

In embodiments, the compound of Formula (VI) is

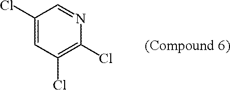

In embodiments, step (i) further comprises a solvent. In embodiments, step (i) occurs in a neat condition.

In embodiments, step (i) comprises contacting about 3-10 equivalents of the compound of Formula (VI) with about 1 equivalent of the activator to obtain about 1 equivalent of the compound of Formula (I) and an amount of the compound of Formula (VI). In embodiments, step (i) comprises contacting about 3 equivalents of the compound of Formula (VI) with about 1 equivalent of the activator.

In embodiments, the process further comprises:

(ii) isolating the compound of Formula (I) obtained in step (i) from the amount of the compound of Formula (VI);

In embodiments, the process further comprises:
(iii) optionally adding about 1 additional equivalent of the compound of Formula (VI) to the remaining amount of the compound of Formula (VI) from step (ii);
(iv) repeating step (i) using the compound of Formula (VI) in step (ii) or step (iii) and about 1 equivalent of the activator; and
(v) optionally repeating steps (ii) to (iv).

In embodiments, step (iii) comprises in total at least about 3 equivalents of the compound of Formula (VI).

In embodiments, the process comprises about 2-8 total cycles to form the compound of Formula (I).

In embodiments, a compound of Formula (I-F) is prepared by a process comprising a step (i-F), wherein said step comprises contacting a compound of Formula (VI) or a salt thereof

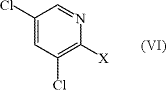

with a fluoride source, and wherein X is a halogen.

In embodiments, a compound of Formula (VI) is

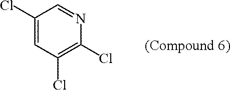

In embodiments, the fluoride source is a fluoride salt, e.g., NaF, LiF, RbF, CsF, and KF.

In embodiments, the fluoride source is KF.

In embodiments, the KF is spray-dried KF.

In embodiments, step (i-F) further comprises a solvent.

In embodiments, the solvent is DMF, DMSO, DMF, DMAc, sulfolane, NMP, MTBE, acetonitrile (ACN), butyronitrile, or a combination thereof.

In embodiments, the solvent is NMP.

In embodiments, step (i-F) further comprises a phase-transfer catalyst.

In embodiments, a phase-transfer catalyst is tetramethylammonium chloride (TMAC), tetramethylammonium fluoride (TMAF), tetrabutylammonium fluoride (TBAF), tetrabutylammonium chloride (TBAC), benzyltriethylammonium chloride (BTEAC), phenyltrimethylammonium chloride (PhTMAC), DMAP-BnCl (salt made from 4-dimethylaminopyridine and benzyl chloride), 18-Crown-6, or a combination thereof.

In embodiments, a phase-transfer catalyst is TMAC.

In embodiments, step (i-F) occurs at a temperature of above about 100° C., of about 110 to about 150° C., of about 130 to about 170° C., or of about 140 to about 160° C. In embodiments, step (i-F) occurs at a temperature of about 150° C.

In embodiments, step (i-F) comprises contacting about 1 equivalent of the compound of Formula (VI) with about 1-3 equivalents of the fluoride source.

In embodiments, step (i-F) comprises contacting about 1 equivalent of the compound of Formula (VI) with about 1.5 equivalents of the fluoride source.

In embodiments, step (i-F) comprises contacting about 1 equivalent of the compound of Formula (VI) with about 0.01-0.5 equivalent of the phase-transfer catalyst.

In embodiments, step (i-F) comprises contacting about 1 equivalent of the compound of Formula (VI) with about 0.1 equivalent of the phase-transfer catalyst.

In embodiments, step (i-F) further comprises:
(ii-F) filtering the reaction mixture obtained from step (i-F); and/or
(iii-F) optionally isolating the compound of Formula (I-F) obtained in step (i-F) or (ii-F) from the amount of the compound of Formula (VI).

In another aspect, disclosed herein is a compound of Formula (I)

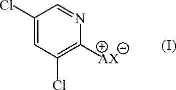

or a tautomer thereof, wherein:
A is an activator; and
X is a halogen.

In embodiments, the activator is DMAP (4-Dimethylaminopyridine), DABCO (1,4-diazabicyclo[2.2.2]octane), DBU (1,8-Diazabicyclo[5.4.0]undec-7-ene), DBN (1,5-Diazabicyclo[4.3.0]non-5-ene), triphenylphosphine, tributylphosphine, or trimethylphosphine.

In embodiments, X is F, Cl or Br.

In embodiments, the compound is

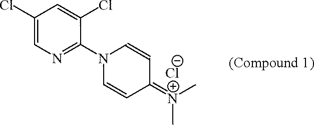 [SMILES: C[N+](C)=c1ccn(-c2ncc(Cl)cc2Cl)cc1 ; [Cl-]]

or a tautomer thereof.

In embodiments, the compound is isolated in solid form.

### DETAILED DESCRIPTION OF THE DISCLOSURE

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

All temperatures are in degrees Celsius (°C) unless otherwise specified.

Unless noted otherwise, all purity and related numeric values (%) are as measured by HPLC.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₈-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a —OH group and may also be referred to herein as a “hydroxyalkyl” group, where the prefix denotes the —OH group and “alkyl” is as described herein. In some embodiments, the alkyl is substituted with a —OR′ group and may also be referred to herein as “alkoxy” group.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a —OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylene: The term “arylene” as used herein refers to an aryl group that is divalent (that is, having two points of attachment to the molecule). Exemplary arylenes include phenylene (e.g., unsubstituted phenylene or substituted phenylene).

Heteroaryl: refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen.

Halogen or Halo: As used herein, the term “halogen” or “halo” means fluorine, chlorine, bromine, or iodine.

Amide: The term “amide” or “amido” refers to a chemical moiety with formula —C(O)N(R′)₂, —C(O)N(R′)—, —NR′C(O)R′, —NR′C(O)N(R′)₂—, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), sulfonyl, amide, or carbonyl group, unless stated other-wise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), amide, or alkyl (“alkylamino”).

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing substituted heterocyclic derivative compounds are known in the art and include, by way of non-limiting example only, the following synthetic methods.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system, e.g., the substitution results in a stable compound (e.g., a compound which does not spontaneously undergo transformation such as by rearrangement, cyclization, elimination, or other reaction). In cases where a specified moiety or group is not expressly noted as being optionally substituted or substituted with any specified substituent, it is understood that such a moiety or group is intended to be unsubstituted.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, arylalkoxy, arylamino, heteroarylamino, heteroaryl, heteroarylalkoxy, heterocycloalkyl, hydroxyalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, amide, cyano, alkoxy, hydroxy, sulfonamide, halo (e.g., —Cl and —Br), nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ and —N(R⁵²)CON(R⁵⁰(R⁵¹), wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Tautomer: refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

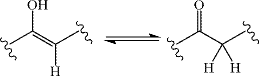

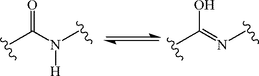

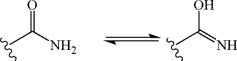

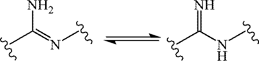

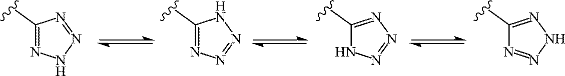

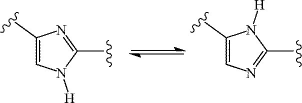

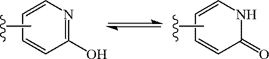

Protecting group: refers to a group of atoms that mask, reduce or prevent the reactivity of the functional group when attached to a reactive functional group in a molecule. Typically, a protecting group may be selectively removed as desired during the course of a synthesis. Examples of protecting groups can be found in Wuts, “Greene’s Protective Groups in Organic Synthesis,” 5th Ed., Wiley (2014), and Harrison et al., Compendium of Synthetic Organic Methods, Vols. 1-8, 1971-1996, John Wiley & Sons, NY. Functional groups that can have a protecting group include, but are not limited to, hydroxy, amino, and carboxy groups. Representative amine protecting groups include, but are not limited to, formyl, acetyl (Ac), trifiuoroacetyl, benzyl (Bn), benzoyl (Bz), carbamate, benzyloxycarbonyl (“CBZ”), p-methoxybenzyl carbonyl (Moz or MeOZ), tertbutoxycarbonyl (“Boc”), trimethylsilyl (“TMS”), 2-trimethylsilyl-ethanesulfonyl (“SES”), trityl and substituted trityl groups, allyloxycarbonyl, 9-fiuorenylmethyloxycarbonyl (“FMOC”), nitro-veratryloxycarbonyl (“NVOC”), p-methoxybenzyl (PMB), tosyl (Ts) and the like.

Solvate: can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

Salt: can include, but are not limited to, salts that retain one or more of the activities and properties of the free acids and bases and that are not undesirable. Illustrative examples of salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

Solvent: can include, but is not limited to, non-polar, polar aprotic, and polar protic solvents. Illustrative examples of non-polar solvents include, but are not limited to, pentane, cyclopentane, hexane, cyclohexane, benzene, toluene, xylene, 1,4-dioxane, chloroform, diethyl ether, and dichloromethane (DCM). Illustrative examples of polar aprotic solvents include, but are not limited to, tetrahydrofuran (THF), ethyl acetate, isopropyl acetate (IPAc), acetone, dimethylformamide (DMF), dimethyl acetamide (DMAc), acetonitrile (MeCN), butyronitrile, dimethyl sulfoxide (DMSO), nitromethane, and propylene carbonate. Illustrative examples of polar protic solvents include, but are not limited to, formic acid, n-butanol, isopropanol (IPA), n-propanol, ethanol, methanol, acetic acid, and water.

Acid: refers to molecules or ions capable of donating a hydron (proton or hydrogen ion H+), or, alternatively, capable of forming a covalent bond with an electron pair (e.g., a Lewis acid). Acids can include, but is not limited to, mineral acids, sulfonic acids, carboxylic acids, halogenated carboxylic acids, vinylogous carboxylic acids, and nucleic acids. Illustrative examples of mineral acids include, but are not limited to, hydrogen halides and their solutions: hydrofluoric acid (HF), hydrochloric acid (HCl), hydrobromic acid (HBr), hydroiodic acid (HI); halogen oxoacids: hypochlorous acid (HClO), chlorous acid (HClO₂), chloric acid (HClO₃), perchloric acid (HClO₄), and corresponding analogs for bromine and iodine, and hypofluorous acid (HFO); sulfuric acid (H₂SO₄); fluorosulfuric acid (HSO₃F); nitric acid (HNO₃); phosphoric acid (H₃PO4); fluoroantimonic acid (HSbF₆); fluoroboric acid (HBF₄); hexafluorophosphoric acid (HPF₆); chromic acid (H₂CrO₄); and boric acid (H₃BO₃). Illustrative examples of sulfonic acids include, but are not limited to, methanesulfonic acid (or mesylic acid, CH₃SO₃H), ethanesulfonic acid (or esylic acid, CH₃CH₂SO₃H), benzenesulfonic acid (or besylic acid, C₆H₅SO₃H), p-toluenesulfonic acid (or tosylic acid, CH₃C₆H₄SO₃H), trifluoromethanesulfonic acid (or triflic acid, CF₃SO₃H), and polystyrene sulfonic acid (sulfonated polystyrene, [CH₂CH(C₆H₄)SO₃H]ₙ). Illustrative examples of carboxylic acids include, but are not limited to, acetic acid (CH₃COOH), citric acid (C₆H₈O7), formic acid (HCOOH), gluconic acid (HOCH₂—(CHOH)₄—COOH), lactic acid (CH₃—CHOH—COOH), oxalic acid (HOOC—COOH), and tartaric acid (HOOC—CHOH—CHOH—COOH). Illustrative examples of halogenated carboxylic acids include, but are not limited to, fluoroacetic acid, trifluoroacetic acid, chloroacetic acid, dichloroacetic acid, and trichloroacetic acid. Illustrative examples of vinylogous carboxylic acids include, but are not limited to, ascorbic acid. Illustrative examples of nucleic acids include, but are not limited to, deoxyribonucleic acid (DNA) and ribonucleic acid (RNA).

Base: refers to molecules or ions capable of accepting protons from a proton donor and/or produce hydroxide ions (OH⁻). Illustrative examples of bases include, but are not limited to, aluminum hydroxide (Al(OH)₃), ammonium hydroxide (NH₄OH), arsenic hydroxide (As(OH)₃), barium hydroxide (Ba(OH)₂), beryllium hydroxide (Be(OH)₂), bismuth(III) hydroxide (Bi(OH)₃), boron hydroxide (B(OH)3), cadmium hydroxide (Cd(OH)₂), calcium hydroxide (Ca(OH)₂), cerium(III) hydroxide (Ce(OH)₃), cesium hydroxide (CsOH), chromium(II) hydroxide (Cr(OH)₂), chromium(III) hydroxide (Cr(OH)₃), chromium(V) hydroxide (Cr(OH)₅), chromium(VI) hydroxide (Cr(OH)₆), cobalt(II) hydroxide (Co(OH)₂), cobalt(III) hydroxide (Co(OH)₃), copper(I) hydroxide (CuOH), copper(II) hydroxide (Cu(OH)₂), gallium(II) hydroxide (Ga(OH)₂), gallium(III) hydroxide (Ga(OH)₃), gold(I) hydroxide (AuOH), gold(III) hydroxide (Au(OH)₃), indium(I) hydroxide (InOH), indium(II) hydroxide (In(OH)₂), indium(III) hydroxide (In(OH)₃), iridium(III) hydroxide (Ir(OH)₃), iron(II) hydroxide (Fe(OH)₂), iron(III) hydroxide (Fe(OH)₃), lanthanum hydroxide (La(OH), lead(II) hydroxide (Pb(OH)₂), lead(IV) hydroxide (Pb(OH)₄), lithium hydroxide (LiOH), magnesium hydroxide (Mg(OH)₂), manganese(II) hydroxide (Mn(OH)₂), manganese(III) hydroxide (Mn(OH)₃), manganese(IV) hydroxide (Mn(OH)₄), manganese(VII) hydroxide (Mn(OH)₇), mercury(I) hydroxide (Hg₂(OH)₂), mercury(II) hydroxide (Hg(OH)₂), molybdenum hydroxide (Mo(OH)₃), neodymium hydroxide (Nd(OH)₃), nickel oxo-hydroxide (NiOOH), nickel(II) hydroxide (Ni(OH)₂), nickel(III) hydroxide (Ni(OH)₃), niobium hydroxide (Nb(OH)₃), osmium(IV) hydroxide (Os(OH)₄), palladium(II) hydroxide (Pd(OH)₂), palladium(IV) hydroxide (Pd(OH)₄), platinum(II) hydroxide (Pt(OH)₂), platinum(IV) hydroxide (Pt(OH)₄), plutonium(IV) hydroxide (Pu(OH)₄), potassium hydroxide (KOH), radium hydroxide (Ra(OH)₂), rubidium hydroxide (RbOH), ruthenium(III) hydroxide (Ru(OH)₃), scandium hydroxide (Sc(OH)₃), silicon hydroxide (Si(OH)₄), silver hydroxide (AgOH), sodium hydroxide (NaOH), strontium hydroxide (Sr(OH)₂), tantalum(V) hydroxide (Ta(OH)₅), technetium(II) hydroxide (Tc(OH)₂), tetramethylammonium hydroxide (C₄H₁₂NOH), thallium(I) hydroxide (TlOH), thallium(III) hydroxide (Tl(OH)₃), thorium hydroxide (Th(OH)₄), tin(II) hydroxide (Sn(OH)₂), tin(IV) hydroxide (Sn(OH)₄), titanium(II) hydroxide (Ti(OH)₂), titanium(III) hydroxide (Ti(OH)₃), titanium(IV) hydroxide (Ti(OH)₄), tungsten(II) hydroxide (W(OH)₂), uranyl hydroxide ((UO₂)₂(OH)₄), vanadium(II) hydroxide (V(OH)₂), vanadium(III) hydroxide (V(OH)₃), vanadium(V) hydroxide (V(OH)₅), ytterbium hydroxide (Yb(OH)₃), yttrium hydroxide (Y(OH)₃), zinc hydroxide (Zn(OH)₂), and zirconium hydroxide (Zr(OH)₄).

Abbreviations and acronyms used herein including the following:

| | |
|---|---|
| Term | Acronym |
| Acetyl | Ac |
| acetonitrile | ACN |
| Aqueous | aq. |
| active pharmaceutical ingredients | API |
| Benzyl | Bn |
| tert-Butyloxycarbonyl | Boc |
| Broad singlet | brs |
| benzyltriethylammonium chloride | BTEAC |
| Doublet | d |
| 1,4-diazabicyclo[2.2. 2]octane | DABCO |
| 1,8-Diazabicyclo[5.4.0]undec-7-ene | DBU |
| 1,5-Diazabicyclo[4.3.0]non-5-ene | DBN |
| Dichloromethane | DCM |
| N,N-Diisopropylethylamine | DIPEA |
| dimethyl acetamide | DMAc |
| 4-Dimethylaminopyridine | DMAP |
| N,N-Dimethylformamide | DMF |
| N,N-dimethylformamide dimethyl acetal | DMF-DMA |
| Dimethylsulfoxide | DMSO |
| Equivalent | eq |
| Electrospray ionization | ESI |
| Ethyl acetate | EtOAc |
| Gram | g |
| Hexanes | Hex |
| High performance liquid chromatography | HPLC |
| Hour | hr |
| isopropyl acetate | IPAc |
| Isopropyl | i-Pr |
| Liquid chromatography-mass spectrometry | LCMS |
| Molarity | M |
| Multiplet | m |
| methylene chloride | MC |
| Megahertz | MHz |
| meta-Chloroperoxybenzoic acid | m-CPBA |
| Methanol | MeOH |
| Milligram | mg |
| Minute | min |
| Milliliter | mL |
| methyl tert-butyl ether | MTBE |
| Normal | N |
| N-methyl-2-pyrrolidone | NMP |
| Nuclear magnetic resonance | NMR |
| Pentet | p |
| Palladium on carbon | Pd/C |
| Petroleum ether | PE |
| Phenyl | Ph |
| phenyltrimethylammonium chloride | PhTMAC |
| Quartet | q |
| Round Bottom Flask | RBF |
| Room temperature | RT |
| Singlet | s |
| Triplet | t |
| tetrabutylammonium chloride | TBAC |
| tetrabutylammonium fluoride | TBAF |
| Triethylamine | TEA |
| Trifluoroacetic acid | TFA |
| Tetrahydrofuran | THF |
| Thin layer chromatography | TLC |
| tetramethylammonium chloride | TMAC |
| tetramethylammonium fluoride | TMAF |

### Methods of the Invention

Vadadustat may be synthesized according to various methods, including methods disclosed in WO 2012/170377 and WO 2019/217550, which are incorporated by reference in their entirety.

Disclosed herein are new methods and processes of preparing intermediates (e.g., a compound of Formula (I), (I-F), (II), or (IV)) or a salt thereof (e.g., any pharmaceutically acceptable salt thereof) useful for the synthesis of vadadustat, as well as new methods and processes for manufacturing vadadustat or a pharmaceutically acceptable salt thereof. Methods described herein can be particularly beneficial for large-scale synthesis of vadadustat.

In certain embodiments, the processes disclosed herein can take place concurrently, in a sequential order as described herein, or in any possible order thereof.

### Compounds of Formula (I) and Methods of Preparation Thereof

Processes described herein can be used to prepare a compound according to Formula (I).

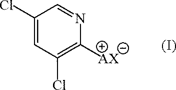

or a tautomer thereof, wherein A is formed from an activator and X is a halogen.

Compounds of Formula (I) include Compound 1, or a tautomer thereof.

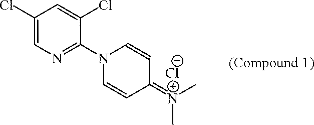 [SMILES: C[N+](C)=c1ccn(-c2ncc(Cl)cc2Cl)cc1 ; [Cl-]]

In embodiments, methods for synthesizing a compound according to Formula (I) (e.g., Compound 1) are unexpectedly efficient and effective for large-scale manufacture of the compound. Accordingly, such methods can be particularly useful for commercial processes for synthesizing compounds such as vadadustat. In particular, methods described herein can achieve high yields of the desired product (e.g., high regioselectivity for functionalization of the pyridyl C2) under relatively mild conditions and/or reduced reaction times.

### Step (i)

In embodiments, methods described herein comprise a step (i). In embodiments, a compound of Formula (I) can be prepared according to Scheme 1.

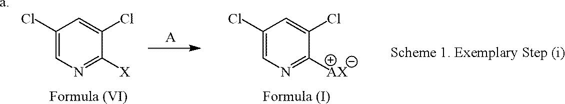

A = Nucleophile Activator such as DMAP, DABCO, pyridine, and the like
X = halogen

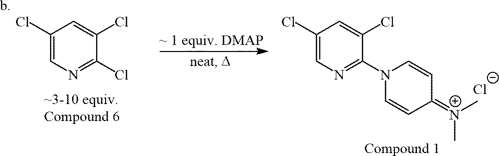

As shown in Scheme 1a, a step (i) can comprise contacting a trihalogenated pyridine compound of Formula (VI) with a nucleophile activator A to form a compound of Formula (I). A further exemplary embodiment of a step (i) is shown in Scheme 1b.

In embodiments, X is F, Cl or Br.

In embodiments, X is Cl, and the compound of Formula (VI) has the following structure,

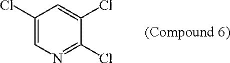

In embodiments, X is Br, and the compound of Formula (VI) has the following structure,

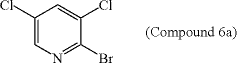

In embodiments, X is F, and the compound of Formula (VI) has the following structure,

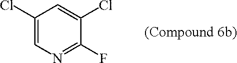

In embodiments, A is a nucleophilic amine, a triarylphosphine, or a trialkylphosphine. In embodiments, A is DMAP (4-dimethylaminopyridine), DABCO (1,4-diazabicyclo[2.2.2]octane), DBU (1,8-Diazabicyclo[5.4.0]undec-7-ene), DBN (1,5-Diazabicyclo[4.3.0]non-5-ene), triphenylphosphine, trimethylphosphine, or tributylphosphine.

In embodiments, A is a nucleophilic amine (e.g., an alkyl amine or aromatic amine). Exemplary nucleophilic amines include but are not limited to methylamine, dimethylamine, trimethylamine, ethylamine, triethylamine, diisopropylamine, piperidine, quinuclidine, DABCO (1,4-diazabicyclo[2.2.2]octane), DBU (1,8-Diazabicyclo[5.4.0]undec-7-ene), DBN (1,5-Diazabicyclo[4.3.0]non-5-ene), aniline, DMAP (4-dimethylaminopyridine), and N,N-Dimethylaniline. In embodiments, A is DMAP, DBU, DBN, or DABCO.

In embodiments, A is a nucleophilic heteroaromatic compound (e.g., A is a nucleophilic pyridyl-, imidazolyl-, or quinolyl-containing compound). Exemplary nucleophilic heteroaromatic compounds include but are not limited to pyridine, DMAP, imidazole, and quinolone.

In embodiments, A is a nucleophilic phosphine (e.g., a triarylphosphine, a trialkylphosphine, or a alkylarylphosphine). Exemplary nucleophilic phosphines include but are not limited to triphenylphosphine, tri(o-tolyl)phosphine, tri(p-tolyl)phosphine, tris(o-methoxyphenyl)phosphine, tris(p-methoxyphenyl)phosphine, tri(2-furyl)phosphine, trimethylphophine, tributylphosphine, tricyclohexylphosphine, cyclohexyldiphenylphosphine, methyldiphenylphosphine, di-tert-butylphenylphosphine, dicyclohexylphenylphosphine, tris(dimethylamino)phosphine. In embodiments, A is triphenylphosphine. In embodiments, A is trimethylphophine or tributylphosphine.

In embodiments, A is a triarylphosphine. Exemplary triarylphosphines include but are not limited to triphenylphosphine, tri(o-tolyl)phosphine, tri(p-tolyl)phosphine, tris(o-methoxyphenyl)phosphine, tris(p-methoxyphenyl)phosphine, and tri(2-furyl)phosphine. In embodiments, A is triphenylphosphine.

In embodiments, A is a trialkylphosphine. Exemplary trialkylphosphines include but are not limited to trimethylphophine, tributylphosphine (e.g., tri-n-butylphosphine or tri-tert-butylphosphine), and tricyclohexylphosphine. In embodiments, A is trimethylphophine. In embodiments, A is tributylphosphine.

In embodiments, A is DMAP, and the compound of Formula (I) has the following structure,

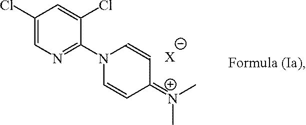

or a tautomer thereof, wherein X is halogen (e.g., F, Br or Cl). In embodiments, the compound of Formula (I) is

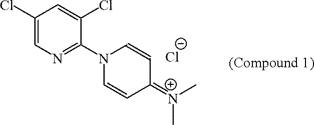 [SMILES: C[N+](C)=c1ccn(-c2ncc(Cl)cc2Cl)cc1 ; [Cl-]]

or a tautomer thereof.

In embodiments, the reaction shown in Scheme 1a occurs in the presence of a solvent.

In embodiments, the reaction shown in Scheme 1a occurs in the absence of a solvent (i.e., occurs in a neat condition). In embodiments, an excess of Formula (VI) is used. In embodiments, a compound according to Formula (VI) (e.g., Compound 6) is present in an amount that is about 2-10 or 3-10 equivalents (e.g., molar equivalents) relative to the nucleophilic activator A (e.g., DMAP). In embodiments, a compound according to Formula (VI) (e.g., Compound 6) is present in an amount that is about 2-5, 2.5-5, 2.5-4, 3-5, or 3-4 equivalents relative to the nucleophilic activator A (e.g., DMAP). In embodiments, a compound according to Formula (VI) (e.g., Compound 6) is present in an amount that is at least about 3 equivalents relative to the nucleophilic activator A (e.g., DMAP). In embodiments, a compound according to Formula (VI) (e.g., Compound 6) is present in an amount that is about 3 equivalents relative to the nucleophilic activator A (e.g., DMAP).

In embodiments, the reaction shown in Scheme 1a occurs at a temperature of about 80° C. to 140° C., about 115° C. to 140° C., or about 130° C. to 140° C. In embodiments, the reaction occurs at a temperature of at least about 80° C. to 140° C. In embodiments, the reaction occurs at a temperature of about 135° C.

In embodiments, a compound according to Formula (VI) (e.g., Compound 6) is allowed to react with the nucleophilic activator A (e.g., DMAP) for about 3 h to about 48 h, about 3 h to about 24 h, or about 3 h to about 5 h. In embodiments, a compound according to Formula (VI) (e.g., Compound 6) is allowed to react with the nucleophilic activator A (e.g., DMAP) for at least about 3 h to about 48 h. In embodiments, a compound according to Formula (VI) (e.g., Compound 6) is allowed to react with the nucleophilic activator A (e.g., DMAP) for less than about 3 h. In embodiments, a compound according to Formula (VI) (e.g., Compound 6) is allowed to react with the nucleophilic activator A (e.g., DMAP) for about 3 h. In embodiments, a compound according to Formula (VI) (e.g., Compound 6) is allowed to react with the nucleophilic activator A (e.g., DMAP) for about 5 h.

In embodiments, the reaction shown in Scheme 1a provides a Compound of Formula (I) (e.g., Compound 1) in about 85% to about 100% yield, about 90% to about 100% yield, or about 95% to about 100% yield. In embodiments, the reaction shown in Scheme 1a provides a Compound of Formula (I) (e.g., Compound 1) in about 94%, 95%, 96%, 97%, 98%, 99%, or 100% yield. In embodiments, the reaction shown in Scheme 1a provides a Compound of Formula (I) (e.g., Compound 1) in about 98% yield.

### Step (ii)

In embodiments, the compound of Formula (I) (e.g., Compound 1) is isolated prior to use in other methods (e.g., as described herein). That is, methods described herein can further comprise a step (ii), where step (ii) comprises isolating the compound of Formula (I) (e.g., Compound 1) obtained in step (i) from the amount of the compound of Formula (VI) (e.g., Compound 6).

In embodiments, a method described herein comprises steps (i) and (ii).

### Steps (iii)-(v)

In embodiments, a process further comprises recycling the excess amount of Formula (VI) (e.g., Compound 6) in further synthetic cycles.

Accordingly, in embodiments, a method further comprises steps (iii) and (iv), wherein step (iii) comprises optionally adding about 1 additional equivalent of the compound of Formula (VI) (e.g., Compound 6) to the remaining amount of the compound of Formula (VI) (e.g., Compound 6) from step (ii); and step (iv) comprises repeating step (i) using the compound of Formula (VI) (e.g., Compound 6) in step (ii) or step (iii) and about 1 equivalent of the activator.

In embodiments, a method comprises a step (v), wherein step (v) comprises optionally repeating steps (ii) to (iv) as described herein.

In embodiments, a method described herein comprises steps (i), (ii), (iii), and (iv). In embodiments, a method described herein comprises steps (i), (ii), and (iv).

In embodiments, a method described herein comprises steps (i), (ii), (iii), (iv), and (v). In embodiments, a method described herein comprises steps (i), (ii), (iv), and (v). In embodiments, the method comprises about 2-10 total cycles for the preparation of a compound of Formula (I) (e.g., Compound 1). In embodiments, step (iii) is repeated only to the extent required to maintain an excess of a compound of Formula (VI) (e.g., Compound 6) (e.g., in order to maintain at least about 3 molar equivalents of a compound of Formula (VI) relative to a compound of Formula (I).

### Exemplary Methods for Synthesis of Compounds of Formula (I)

In embodiments, a compound of Formula (I) (e.g., Compound 1) can be prepared by a process (e.g., a process comprises one or more steps of step (i) to (v)) according to Scheme 2.

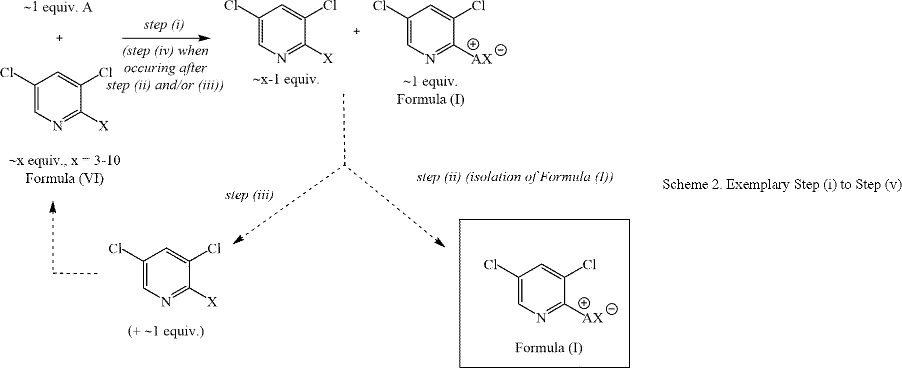

A = Nucleophile Activator such as DMAP, DABCO, pyridine, and the like
X = halogen

In Scheme 2, a step (v) can comprise repeating steps (ii) and (iv) and/or repeating steps (ii), (iii), and (iv).

In embodiments, a method comprises step (i), including any embodiment described herein.

In embodiments, a method comprises steps (i) and (ii), including any combination of embodiments described herein.

In embodiments, a method comprises steps (i), (ii), and (iv), including any combination of embodiments described herein.

In embodiments, a method comprises steps (i), (ii), (iii), and (iv), including any combination of embodiments described herein.

In embodiments, a method comprises steps (i), (ii), (iii), (iv), and (v). In embodiments, step (v) comprises repeating steps (ii), (iv), and optionally (iii) about 1-8 times.

In embodiments, step (iii) comprises in total at least about 3 equivalents of the compound of Formula (VI) (e.g., Compound 6). In embodiments, step (iii) comprises in total at least about 2-5, 2.5-5, 2.5-4, 3-5, or 3-4 equivalents of the compound of Formula (VI) (e.g., Compound 6).

In embodiments, a process described herein comprises about 2-8 total cycles to form the compound of Formula (I) (e.g., Compound 1). In embodiments, a process described herein comprises more than 8 total cycles to form the compound of Formula (I) (e.g., Compound 1).

In embodiments, a method further comprises step (a) in any embodiment as described herein. In embodiments, a method further comprises steps (a) and (b) in any combination of embodiments as described herein. In embodiments, a method further comprises steps (a), (b), and (c) in any combination of embodiments as described herein.

### Compounds of Formula (I-F) and Methods of Preparation Thereof

Processes described herein can be used to prepare a compound according to Formula (I-F).

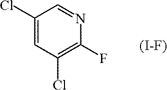

### Step (I-F)

In embodiments, methods described herein comprise a step (i-F). For example, a compound of Formula (I-F) can be prepared according to Scheme 1-F.

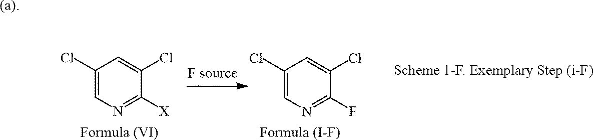

X = halogen

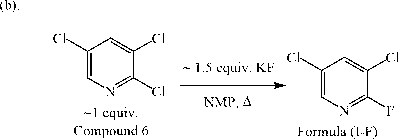

As shown in Scheme 1-F(a), a step (i-F) can comprise contacting a trihalogenated pyridine compound of Formula (VI) with a fluoride source to form a compound of Formula (I-F). A further exemplary embodiment of a step (i-F) is shown in Scheme 1-F(b).

In embodiments, X is Cl or Br.

In embodiments, X is Cl, and the compound of Formula (VI) has the following structure,

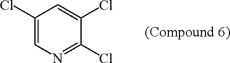

In embodiments, X is Br, and the compound of Formula (VI) has the following structure,

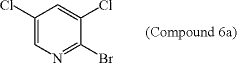

In embodiments, a fluoride source includes but is not limited to an inorganic fluoride salt (e.g., CaF₂, RbF, CsF, LiF, NaF, KF, or AgF), a tetraalkylammonium fluoride (e.g., tetra-n-butylammoniumfluoride, tetramethylammonium fluoride), an antimony fluoride (e.g., antimony(III) fluoride, or antimony(V) fluoride), a HF-amine complex (e.g., HF-2,4,6-collidine complex, HF-pyridine complex, tetra-n-butylammonium dihydrogentrifluoride, or triethylamine trihydrofluoride), a fluoroborate (e.g., tetrafluoroboric acid, hexafluorophosphoric acid, nitrosonium tetrafluoroborate, or silver tetrafluoroborate), a sulfur fluoride (e.g., diethylaminosulfur trifluoride, or morpholinosulfur trifluoride), and a hydrogen fluoride equivalent (e.g., cyanuric fluoride, Ishikawa’s reagent, Yarovenko’s reagent, or Gingras’ reagent).

In embodiments, a fluoride source is an inorganic fluoride salt (e.g., CaF₂, RbF, CsF, LiF, NaF, KF, or AgF).

In embodiments, a fluoride source is KF. In embodiments, a KF is crystalline solid (e.g., about 0.1 mm). In embodiments, a KF is fine powder KF. In embodiments, a KF is grounded KF (e.g., hand- grounded KF). In embodiments, a KF is spray-dried KF (e.g., 75-150 microns, moisture < 0.2%).

In embodiments, an excess of fluoride source (e.g., KF) is used. In embodiments, a fluoride source (e.g., KF) is present in an amount that is about 1-3 equivalents (e.g., molar equivalents) relative to a compound according to Formula (VI) (e.g., Compound 6). In embodiments, a fluoride source (e.g., KF) is present in an amount that is higher than about 1-3 (e.g., about 3-10) equivalents (e.g., molar equivalents) relative to a compound according to Formula (VI) (e.g., Compound 6). In embodiments, a fluoride source (e.g., KF) is present in an amount that is about 1-1.5, 1.5-2, 2-2.5, 2.5-3, 1-2, or 2-3 equivalents (e.g., molar equivalents) relative to a compound according to Formula (VI) (e.g., Compound 6). In embodiments, a fluoride source (e.g., KF) is present in an amount that is about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 equivalents (e.g., molar equivalents) relative to a compound according to Formula (VI) (e.g., Compound 6). In embodiments, a fluoride source (e.g., KF) is present in an amount that is about 1.5 equivalents (e.g., molar equivalents) relative to a compound according to Formula (VI) (e.g., Compound 6).

In embodiments, the exemplary reaction shown in Scheme 1-F occurs in the absence of a phase-transfer catalyst.

In embodiments, the exemplary reaction shown in Scheme 1-F occurs in the presence of a phase-transfer catalyst.

Exemplary phase-transfer catalysts include but are not limited to ammonium salts (e.g., tetraalkylammonium salts such as tetramethylammonium chloride), phosphonium salts, and crown ethers (e.g., 18-Crown-6).

In embodiments, a phase-transfer catalyst is an ammonium salt. Exemplary ammonium salt phase-transfer catalysts include but are not limited to tetramethylammonium chloride, tetramethylammonium iodide, tetramethylammonium bromide, tetramethylammonium fluoride, tetramethylammonium tetrafluoroborate, tetraethylammonium fluoride, tetraethylammonium chloride, tetraethylammonium bromide, tetraethylammonium iodide, tetrabutylammonium fluoride, tetrabutylammonium chloride, tetrabutylammonium bromide, tetrabutylammonium iodide, tetra-n-octylammonium iodide, benzyltriethylammonium chloride, phenyltrimethylammonium chloride, triethylmethylammonium tetrafluoroborate, and dimethyldioctylammonium bromide. In embodiments, a phase-transfer catalyst is tetramethylammonium chloride (TMAC).

In embodiments, a phase-transfer catalyst is a phosphonium salt. Exemplary phosphonium salt phase-transfer catalysts include but are not limited to tributyl(cyanomethyl)phosphonium chloride, tetraethylphosphonium hexafluorophosphate, tetraethylphosphonium tetrafluoroborate, tetrabutylphosphonium bromide, tetrabutylphosphonium chloride, tetrabutylphosphonium tetrafluoroborate, tributyl-n-octylphosphonium bromide, tributylhexadecylphosphonium bromide, tetrabutylphosphonium hexafluorophosphate, tetrabutylphosphonium tetraphenylborate, tetraphenylphosphonium chloride, and tetraphenylphosphonium bromide.

In embodiments, a phase-transfer catalyst is a crown ether. Exemplary crown ether phase-transfer catalysts include but are not limited to 12-crown-4, 15-crown-5, 18-Crown-6, and 21-crown-7.

In embodiments, the reaction occurs in the presence of tetramethylammonium chloride (TMAC), tetramethylammonium fluoride (TMAF), tetrabutylammonium fluoride (TBAF), tetrabutylammonium chloride (TBAC), benzyltriethylammonium chloride (BTEAC), phenyltrimethylammonium chloride (PhTMAC), DMAP-BnCl (salt made from 4-dimethylaminopyridine and benzyl chloride), or 18-Crown-6, or a combination thereof.

In embodiments, the reaction occurs in the presence of tetramethylammonium chloride (TMAC).

In embodiments, a phase-transfer catalyst (e.g., TMAC) is present in an amount that is about 0.01-0.5 equivalents (e.g., molar equivalents) relative to a compound according to Formula (VI) (e.g., Compound 6). In embodiments, a phase-transfer catalyst (e.g., TMAC) is present in an amount that is higher than about 0.5 equivalents (e.g., molar equivalents) relative to a compound according to Formula (VI) (e.g., Compound 6). In embodiments, a phase-transfer catalyst (e.g., TMAC) is present in an amount that is about 0.01-0.1, 0.1-0.2, 0.2-0.3, 0.3-0.4, or 0.4-0.5 equivalents (e.g., molar equivalents) relative to a compound according to Formula (VI) (e.g., Compound 6). In embodiments, a phase-transfer catalyst (e.g., TMAC) is present in an amount that is about 0.01, 0.05, 0.1, 0.15, 0.2, 0.25, 0.3, 0.35 0.4, 0.45, or 0.5 equivalents (e.g., molar equivalents) relative to a compound according to Formula (VI) (e.g., Compound 6). In embodiments, a phase-transfer catalyst (e.g., TMAC) is present in an amount that is about 0.1 equivalents (e.g., molar equivalents) relative to a compound according to Formula (VI) (e.g., Compound 6).

In embodiments, the reaction shown in Scheme 1-F occurs in the absence of a solvent (i.e., occurs in a neat condition).

In embodiments, the reaction shown in Scheme 1-F occurs in the presence of a solvent (e.g., a solvent as described herein).

In embodiments, the solvent is an ester solvent (e.g., an ester solvent as described herein). In embodiments, the solvent is an aprotic solvent. In embodiments, the solvent is a combination of two or more solvents (e.g., two or more solvents as described herein). In embodiments, an aprotic solvent is methyl tert-butyl ether (MTBE), N-methyl-2-pyrrolidone (NMP), acetonitrile (ACN), acetone, heptane, methylene chloride (MC), trigyme, tetrahydrofuran (THF), dimethylformamide (DMF), dimethyl sulfoxide (DMSO), or dimethyl acetamide (DMAc). In embodiments, the reaction occurs in the presence of NMP.

In embodiments, the reaction shown in Scheme 1-F occurs in a solvent with a concentration of about 1 to 6 volumes (1 volume = 1 mL solvent per 1 g a compound of Formula (VI) (e.g., Compound 6)). In embodiments, the reaction occurs in a solvent with a concentration of less than 1 volumes. In embodiments, the reaction occurs in a solvent with a concentration of more than 6 volumes. In embodiments, the reaction occurs in a solvent with a concentration of about 1, 2, 3, 3.5, 4, 4.5, 5, 5.5, or 6 volumes. In embodiments, the reaction occurs in a solvent with a concentration of about 3 volumes.

In embodiments, the reaction shown in Scheme 1-F occurs at a temperature of at least about 100° C. In embodiments, the reaction occurs at a temperature of lower than 100° C. In embodiments, the reaction occurs at a temperature of about 115 to 150° C., about 130° C. to 170° C., or about 140° C. to 160° C. In embodiments, the reaction occurs at a temperature of about 115, 130, 135, 140, 145, 150, 155, 160, 165, or 170° C. In embodiments, the reaction occurs at a temperature of about 150° C.

In embodiments, a compound according to Formula (VI) (e.g., Compound 6) is allowed to react with a fluoride source (e.g., KF) for about 3 h to about 24 h, about 3 h to 12 h, about 3 h to about 8 h, or about 3 h to about 5 h. In embodiments, a compound according to Formula (VI) (e.g., Compound 6) is allowed to react with a fluoride source (e.g., KF) for at least about 3 h to about 24 h. In embodiments, a compound according to Formula (VI) (e.g., Compound 6) is allowed to react with a fluoride source (e.g., KF) for about 3, 4, 5, 6, 7, 8, 12, 15, 18, or 24 h. In embodiments, a compound according to Formula (VI) (e.g., Compound 6) is allowed to react with a fluoride source (e.g., KF) for no more than about 3, 4, 5, 6, 7, 8, 12, 15, 18, or 24 h. In embodiments, a compound according to Formula (VI) (e.g., Compound 6) is allowed to react with a fluoride source (e.g., KF) for about 4 h.

In embodiments, the reaction shown in Scheme 1-F(a) provides a Compound of Formula (I-F) in about 70% to about 100% HPLC yield, about 80 to about 100% HPLC yield, or about 80% to about 90% HPLC yield.

In embodiments, the reaction mixture obtained from step (i-F) is used directly in other methods (e.g., to prepare a compound of Formula (II), (IVa), or (IV) as described herein).

In embodiments, a method described herein comprises step (i-F).

### Step (II-F)

In embodiments, the compound of Formula (I-F) is not isolated prior to use in other methods (e.g., as described herein).

In embodiments, the reaction mixture from step (i-F) is filtered prior to use in other methods (e.g., as described herein). That is, methods described herein can further comprise a step (ii-F), where step (ii-F) comprises filtering the reaction mixture from step (i-F), and obtaining the filtrate comprising compound of Formula (I-F). In embodiments, a filtration step comprises washing the filter cake with a solvent (e.g., a solvent as described herein such as MTBE). That is, methods described herein can further comprise a step (ii-F), where step (ii-F) comprises filtering the reaction mixture from step (i-F), washing the filter cake with a solvent (e.g., a solvent as described herein such as MTBE), and obtaining the combined filtrate comprising compound of Formula (I-F).

### Step (III-F)

In embodiments, the compound of Formula (I-F) is isolated prior to use in other methods (e.g., as described herein). That is, methods described herein can further comprise a step (iii-F), where step (iii-F) comprises isolating the compound of Formula (I-F) obtained in step (i-F) or step (ii-F).

In embodiments, a method described herein comprises steps (i-F) and (ii-F). In embodiments, a method described herein comprises steps (i-F) and (iii-F). In embodiments, a method described herein comprises steps (i-F), (ii-F) and (iii-F).

In embodiments, a method further comprises step (a-F) in any embodiment as described herein. In embodiments, a method further comprises steps (a-F) and (b) in any combination of embodiments as described herein. In embodiments, a method further comprises steps (a-F), (b), and (c) in any combination of embodiments as described herein.

### Compounds of Formula (II) and Methods of Preparation Thereof

Processes described herein can be used to prepare a compound according to Formula (II).

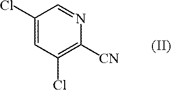

### Step (a)

In embodiments, methods described herein comprise a step (a). In embodiments, a compound of Formula (II) can be prepared according to Scheme 3.

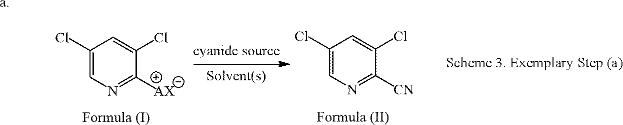

A = Nucleophile Activator such as DMAP, DABCO, pyridine, and the like
X = halogen
cyanide source is NaCN, KCN, TMSCN, Zn(CN)2, Cu(CN)₂, or the like

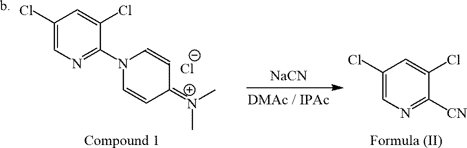

As shown in Scheme 3a, a step (a) can comprise contacting a compound of Formula (I) (e.g., Compound 1) with a cyanide source to form a compound of Formula (II). The contacting may be done in the presence of one or more solvents. An additional exemplary embodiment of a step (a) is shown in Scheme 3b.

In embodiments, a compound of Formula (I) as shown in Scheme 3a is Compound 1.

In embodiments, a compound of Formula (I) is prepared by a method as described herein.

In embodiments, a compound of Formula (I) (e.g., Compound 1) can be contacted with about 0 to 5, 1 to 3, or 1.5 to 2 equivalents (e.g., molar equivalents) of a cyanide source (e.g., NaCN) to form a compound of Formula (II). In embodiments, a compound of Formula (I) (e.g., Compound 1) can be contacted with at least about 0 to 5 equivalents (e.g., molar equivalents) of a cyanide source (e.g., NaCN) to form a compound of Formula (II). In embodiments, a compound of Formula (I) can be contacted with about 1, 1.5, 2, 2.5, 3, 3.5, 4, 4.5, or 5 equivalents (e.g., molar equivalents) of a cyanide source (e.g., NaCN) to form a compound of Formula (II). In embodiments, a compound of Formula (I) (e.g., Compound 1) can be contacted with about 1.5 equivalents (e.g., molar equivalents) of a cyanide source (e.g., NaCN) to form a compound of Formula (II).

In embodiments, a cyanide source is an inorganic cyanide salt. In embodiments, a cyanide source is NaCN, KCN, Zn(CN)₂, or CuCN. In embodiments, a cyanide source is an organosilicon cyanide compound. In embodiments, a cyanide source is TMSCN (trimethylsilyl cyanide). In embodiments, a cyanide source is NaCN.

In embodiments, the reaction shown in Scheme 3 occurs in the presence of a solvent. In embodiments, the solvent is an ester solvent. In embodiments, an ester solvent is isopropyl acetate (IPAc) or ethyl acetate. In embodiments, the solvent is an aprotic solvent. In embodiments, an aprotic solvent is trigyme, tetrahydrofuran (THF), dimethylformamide (DMF), dimethyl sulfoxide (DMSO), or dimethyl acetamide (DMAc).

In embodiments, the solvent is isopropyl acetate (IPAc).

In embodiments, the solvent is a combination of two or more solvents. In embodiments, the solvent is a combination of an ester solvent (e.g., IPAc) and an aprotic solvent (e.g., DMAc). In embodiments, the solvent combination comprises IPAc and DMAc. In embodiments, IPAc and DMAc are present in a ratio (e.g., v/v) ranging from about 19:1 to 1:1 IPAc:DMAc. In embodiments, IPAc and DMAc are present in a ratio (e.g., v/v) higher than 19:1 IPAc:DMAc. In embodiments, IPAc and DMAc are present in a ratio (e.g., v/v) lower than 1:1 IPAc:DMAc. In embodiments, IPAc and DMAc are present in a ratio (e.g., v/v) of about 19:1 IPAc:DMAc. In embodiments, the IPAc and DMAc are present in a ratio (e.g., v/v) of about 19:1. In embodiments, the IPAc and DMAc are present in a ratio (e.g., v/v) of about 6.5:0.5.

In embodiments, the reaction shown in Scheme 3 occurs in a solvent with a concentration of about 5 to 10 volumes (1 volume = 1 mL solvent per 1 g a compound of Formula (I)). In embodiments, the reaction occurs in a solvent with a concentration of less than 5 volumes. In embodiments, the reaction occurs in a solvent with a concentration of more than 10 volumes. In embodiments, the reaction occurs in a solvent with a concentration of about 5, 6, 7, 8, 9, or 10 volumes. In embodiments, the reaction occurs in a solvent with a concentration of about 7 volumes. In embodiments, the reaction occurs in a solvent with a concentration of about 10 volumes.

In embodiments, the reaction shown in Scheme 3 occurs at a temperature of about 50° C. to about 140° C., about 70° C. to about 110° C., about 80° C. to about 100° C., or about 85° C. to about 95° C. In embodiments, the reaction occurs at a temperature of at least about 50° C. to about 140° C. In embodiments, the reaction occurs at a temperature of lower than about 50° C. In embodiments, the reaction occurs at a temperature of about 90° C.

In embodiments, a method comprises step (a) and any combination of steps (i)-(v) described herein, including any combination of any embodiments described herein.

### Step (A-F)

In embodiments, methods described herein comprise a step (a-F). For example, a compound of Formula (II) can be prepared according to Scheme 3-F.

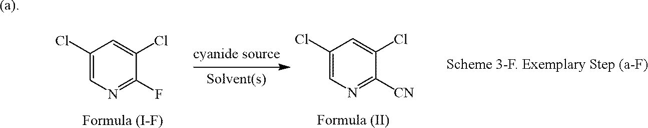

cyanide source is NaCN, KCN, TMSCN, Zn(CN)₂, Cu(CN)₂, or the like

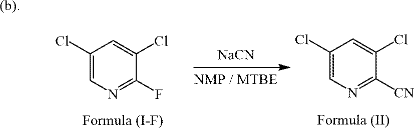

As shown in Scheme 3-F(a), a step (a-F) can comprise contacting a compound of Formula (I-F) with a cyanide source to form a compound of Formula (II). The contacting may be done in the presence of one or more solvents. An additional exemplary embodiment of a step (a-F) is shown in Scheme 3-F(b).

In embodiments, a compound of Formula (I-F) is prepared by a method as described herein.

In embodiments, a compound of Formula (I-F) can be contacted with about 0 to 5, 1 to 3, or 1.5 to 2 equivalents (e.g., molar equivalents) of a cyanide source (e.g., NaCN) to form a compound of Formula (II). In embodiments, a compound of Formula (I-F) can be contacted with at least about 0 to 5 equivalents (e.g., molar equivalents) of a cyanide source (e.g., NaCN) to form a compound of Formula (II). In embodiments, a compound of Formula (I-F) can be contacted with about 1, 1.5, 2, 2.5, 3, 3.5, 4, 4.5, or 5 equivalents (e.g., molar equivalents) of a cyanide source (e.g., NaCN) to form a compound of Formula (II). In embodiments, a compound of Formula (I-F) can be contacted with about 1.5 equivalents (e.g., molar equivalents) of a cyanide source (e.g., NaCN) to form a compound of Formula (II).

In embodiments, a cyanide source is an inorganic cyanide salt. In embodiments, a cyanide source is NaCN, KCN, Zn(CN)₂, or CuCN. In embodiments, a cyanide source is an organosilicon cyanide compound. In embodiments, a cyanide source is TMSCN (trimethylsilyl cyanide). In embodiments, a cyanide source is NaCN.

In embodiments, the reaction shown in Scheme 3-F occurs in the presence of a solvent. In embodiments, the solvent is an ester solvent (e.g., an ester solvent as described herein). In embodiments, the solvent is an aprotic solvent. In embodiments, an aprotic solvent is methyl tert-butyl ether (MTBE), N-methyl-2-pyrrolidone (NMP), acetonitrile (ACN), acetone, heptane, methylene chloride (MC), trigyme, tetrahydrofuran (THF), dimethylformamide (DMF), dimethyl sulfoxide (DMSO), or dimethyl acetamide (DMAc). In embodiments, the solvent is N-methyl-2-pyrrolidone (NMP).

In embodiments, the solvent is a combination of two or more solvents (e.g., two or more solvents as described herein). In embodiments, the solvent is a combination of two or more aprotic solvents (e.g., two or more aprotic solvents as described herein). In embodiments, the solvent combination comprises NMP and another aprotic solvents (e.g., an aprotic solvent as described herein). In embodiments, the solvent combination comprises NMP and MTBE. In embodiments, NMP and MTBE are present in a ratio (e.g., v/v) ranging from about 1:3 to 6:1 NMP:MTBE. In embodiments, NMP and MTBE are present in a ratio (e.g., v/v) higher than about 6:1 NMP:MTBE. In embodiments, NMP and MTBE are present in a ratio (e.g., v/v) lower than about 1:3 NMP:MTBE. In embodiments, NMP and MTBE are present in a ratio (e.g., v/v) of about 3:2 NMP:MTBE.

In embodiments, the reaction shown in Scheme 3-F occurs in the presence of a solvent that comprises water. In embodiments, the solvent comprises about 1% to 5%, or about 2% to 4% water. In embodiments, the solvent comprises no more than about 1% water. In embodiments, the solvent comprises at least about 5% water. In embodiments, the solvent comprises about 1%, 1.5%, 2%, 2.5%, 3%, 3.5%, 4%, 4.5% or 5% water. In embodiments, the solvent comprises about 3% water.

In embodiments, the reaction shown in Scheme 3-F occurs in a solvent with a concentration of about 2 to 10, about 4 to 8, about 2 to 4, about 4 to 6, or about 6 to 10 volumes (1 volume = 1 mL solvent per 1 g a compound of Formula (I-F)). In embodiments, the reaction occurs in a solvent with a concentration of no more than 2 volumes. In embodiments, the reaction occurs in a solvent with a concentration of at least 10 volumes. In embodiments, the reaction occurs in a solvent with a concentration of about 2, 3, 3.5, 4, 4.5, 5, 5.5, 6, 6.5, 7, 8, 9 or 10 volumes. In embodiments, the reaction occurs in a solvent with a concentration of about 5 volumes.

In embodiments, the reaction shown in Scheme 3-F occurs at a temperature of about 25 to 100° C., about 35 to 100° C., about 45 to 100° C., about 55 to 100° C., about 55 to 65° C., about 50 to 70° C., or about 45 to 75° C. In embodiments, the reaction occurs at a temperature of higher than about 100° C. In embodiments, the reaction occurs at a temperature of lower than about 25° C. In embodiments, the reaction occurs at a temperature of about 25, 30, 35, 40, 45, 50, 55, 60, 65, 70, 75, 80, 85, 90, 95, or 100° C. In embodiments, the reaction occurs at a temperature of about 60 or 80° C. In embodiments, the reaction occurs at a temperature of about 60° C.

In embodiments, a method comprises step (a-F) and any combination of steps (iF)-(iii-F) described herein, including any combination of any embodiments described herein.

### Compounds of Formula (IV) and Methods of Preparation Thereof

Processes described herein can be used to prepare a compound according to Formula (IV).

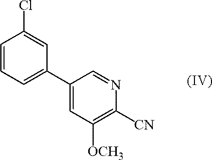 [SMILES: COc1cc(-c2cccc(Cl)c2)cnc1C#N]

### Steps (b) and (c)

In embodiments, methods described herein comprise a step (b) and/or step (c). In embodiments, a compound of Formula (IV) can be prepared according to Scheme 4.

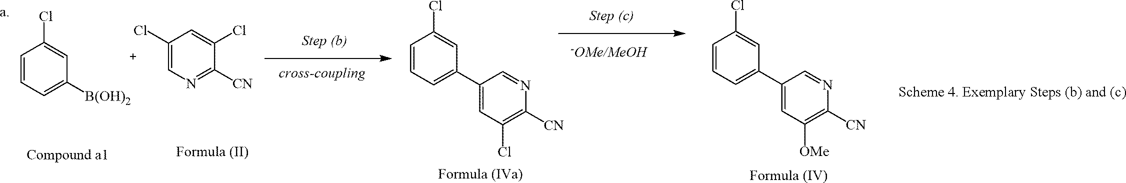

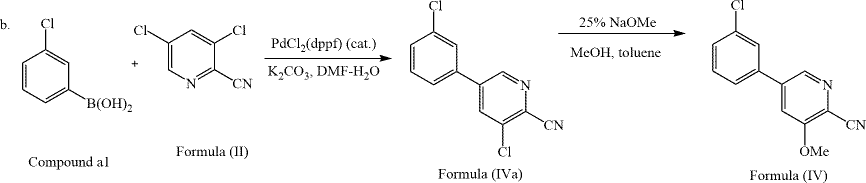

As shown in Scheme 4a, (3-chlorophenyl)boronic acid (Compound a1) can be coupled to a compound of Formula (II) using a palladium catalyst to provide a compound of Formula (IVa). Then a compound of Formula (IVa) can be treated with sodium methoxide to provide a compound of Formula (IV). A further exemplary embodiment of steps (b) and/or (c) is provided in Scheme 4b.

In embodiments, a method comprises steps (a) and (b) in any combination of embodiments described herein. In embodiments, a method comprises steps (a-F) and (b) in any combination of embodiments described herein. In embodiments, a method comprises steps (a), (b), and (c) in any combination of embodiments described herein. In embodiments, a method comprises steps (a-F), (b), and (c) in any combination of embodiments described herein. In embodiments, a method further comprises any combination of steps (i)-(v) described herein, including any combination of any embodiments described herein. In embodiments, a method further comprises any combination of steps (i-F)-(iii-F) described herein, including any combination of any embodiments described herein.

In embodiments, a compound of Formula (II) is prepared as described herein.

### Compounds of Formula (V) (Vadadustat) and Methods of Preparation Thereof

Processes described herein can be used to prepare a compound of Formula (V) (vadadustat).

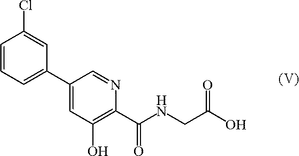 [SMILES: O=C(O)CNC(=O)c1ncc(-c2cccc(Cl)c2)cc1O]

For example, processes described herein can used to prepare vadadustat according to methods described in WO 2019/217550, which is hereby incorporated by reference. For example, a method for preparing compound (V) can comprise any or all of the following steps:
Step (d1). Hydrolysis of a compound of Formula (IV) to form a carboxylic acid (Compound a2) from the nitrile moiety;
Step (d2). Peracylation of Compound a2 (e.g., contacting Compound a2 with pivaloyl chloride to form a Compound a3);
Step (e). Contacting a peracylated compound (e.g., Compound a3) with a glycine methyl ester to form a Compound a4; and
Step (f). Hydrolysis of the product of step (e) to form a compound of Formula (V) (vadadustat).

In embodiments, vadadustat can be prepared according to Scheme 5, where a compound of Formula (IV) is prepared as described herein.

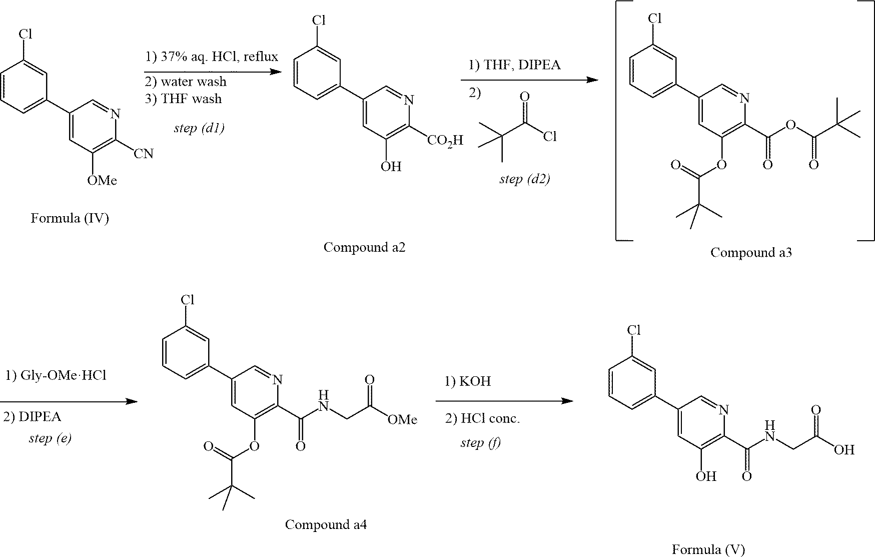

As shown in Scheme 5, a compound of Formula (IV) can be converted to carboxylic acid a2 using hydrochloric acid. Then, Compound a2 can be treated with pivolyl chloride to provide mixed anhydride a3, which can be converted to Compound a4 upon reaction with glycine methyl ester hydrochloride (a small amount of de-protected version of Compound a4 at the pyridin-3-ol position may be formed, which can be converted directly to vadadustat without further purification). Compound a4 can be converted to vadadustat under basic conditions (e.g., KOH).

Processes described herein also can used to prepare vadadustat according to methods described in WO 2012/170377, which is hereby incorporated by reference. For example, a method for preparing compound (V) can comprise any or all of the following steps:
Step (d). Hydrolysis of a compound of Formula (IV) to form a carboxylic acid (Compound a2) from the nitrile moiety;
Step (e). Contacting the product of step (d) (Compound a2) with a glycine methyl ester to form a Compound a5; and
Step (f). Hydrolysis of the product of step (e) to form a compound of Formula (V) (vadadustat).

In embodiments, vadadustat can be prepared according to Scheme 6, where a compound of Formula (IV) is prepared as described herein.

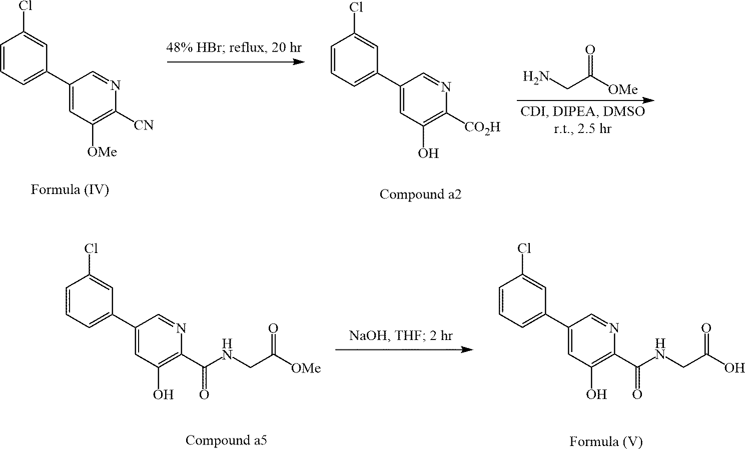

As shown in Scheme 6, a compound of Formula (IV) can be converted to carboxylic acid a2 using hydrobromic acid. Then, Compound a2 can be coupled with a glycine methyl ester to form Compound a5, which in turn can be converted to vadadustat under basic conditions (e.g., NaOH/THF).

### EXMPLIFICATION

The following examples illustrate some embodiments and aspects of the invention. It will be apparent to those skilled in the relevant art that various modifications, additions, substitutions, and the like can be performed without altering the spirit or scope of the invention, and such modifications and variations are encompassed with invention as defined in the claims which follow. The invention disclosed herein is further illustrated by the following examples which in no way should be construed as being limiting.

### Example A: Preparation of DMAP Salt

### Example A-1: Preparation of DMAP Salt Intermediate (Neat Condition)

2,3,5-Trichloropyridine (TCP, 900 g, 3.0eq) and 4-dimethylaminopyride (DMAP, 200 g, 1.0eq) were charged to a 5-L 2 neck RBF (round bottom flask) fitted with a condenser, mechanical stirrer and oil bath. The reaction was heated to 130° C. ± 5° C. with stirring. The reaction was monitor by 1H NMR with sampling every hour for consumption of TCP. After complete TCP consumption the reaction was cooled to 70° C. and isopropyl acetate (IPAc, 2000 mL, 4 volumes (based on product formation at 100%) was charged to the mixture. The mixture was allowed to agitate at 70° C. for 1 hour. The reaction was cooled to 60° C. at which time it was filtered, and the resulting white solids were washed with IPAc (2 × 500 mL, 2 × 1 vol). The solid was dried in a vacuum oven at 30° C. with an N2 bleed to afford an off-white solid (487.0 g, 97.5% yield). 1H NMR (300 MHz, CDCl3) δ 8.50-8.40 (dd, 3H), 7.98 (d, 1H), 7.48-7.46 (d, 2H), 3.42 (s, 6H).

### Example A-2: Preparation of DMAP Salt Intermediate (Xylene Condition)

2,3,5-Trichloropyridine (TCP, 50 g, 1.0eq), 4-dimethylaminopyride (DMAP, 35.15 g, 1.05eq), and xylene (100 mL, 2 vol) were charged to a 500-mL 2 neck RBF fitted with a condenser, mechanical stirrer and oil bath. The reaction was heated to 140° C. ± 5° C. with stirring. The reaction was monitor by 1H NMR for consumption of TCP. After complete TCP consumption, 40 hours of reaction time, the reaction was cooled to 20° C. ± 5° C. and filtered. The resulting solids were washed with toluene (200 mL, 1 × 4 vol) followed by hexanes (100 mL, 1 × 2 vol). The solid was dried in a vacuum oven at 30° C. with an N2 bleed to afford an off-white solid (79.86 g, 95.66% yield). 1H NMR (300 MHz, CDCl3) δ 8.50-8.40 (dd, 3H), 7.98 (d, 1H), 7.48-7.46 (d, 2H), 3.42 (s, 6H).

### Example A-3: Preparation of DMAP Salt Intermediate (Butyronitrile Condition)

2,3,5-Trichloropyridine (TCP, 20 g, 1.0eq), 4-dimethylaminopyride (DMAP, 14.06 g, 1.05eq), and butyronitrile (150 mL, 7.5 vol) were charged to a 500-mL 2 neck RBF fitted with a condenser, mechanical stirrer and oil bath. The reaction was heated to 120° C. ± 5° C. with stirring. The reaction was monitor by ¹H NMR for consumption of TCP. After complete TCP consumption, 47 hours of reaction time, the reaction was cooled to 20° C. ± 5° C. and filtered. The resulting solids were washed with butyronitrile (10 mL, 1 × 0.5 vol). The solid was dried in a vacuum oven at 30° C. with an N₂ bleed to afford an off-white solid (23.33 g, 64.6% yield). ¹H NMR (300 MHz, CDCl3) δ 8.5-8.40 (dd, 3H), 7.98 (d, 1H), 7.48-7.46 (d, 2H), 3.42 (s, 6H).

### Example B: Preparation of 3,5-Dichloropicolmonitrile

### Example B-1: Preparation of 3,5-Dichloropicolinonitrile (IPAc Condition)

DMAP salt from last step (50.0 g, 1.0eq) was charged to a 1-L 2 neck RBF fitted with a condenser, mechanical stirrer and oil bath. IPAc (500 mL, 10 vol) was charged. The reaction was agitated at room temperature. Sodium cyanide (12.07 g, 24.6 mmol, 1.5eq) was charged. The reaction was heated to 80° C. ± 5° C. with stirring for 10 hours. The reaction was monitored by HPLC analysis for % conversion (98.1%). The reaction was cooled to 40° C. and concentrated under vacuum to 100 mL (2 vol). The reaction was cooled to 25° C. ± 5° C. and water was charged (250 mL, 5 vol) followed by toluene (250 mL, 5 vol). After 30 minutes stirring was stopped, and the phases allowed to separate. The organic phase was retained and the aqueous phase was extracted with toluene (150 mL, 1 × 3 vol). The organic phases were combined and washed with 10% NaClO solution (100 mL, 1 × 2 vol). The organic phase was washed with 2N HCl solution (100 mL, 1 × 2 vol) followed by water (100 mL, 1 × 2 vol). The organic phase was then decolorized with activated carbon or charcoal and then filtered. The filter cake was washed with toluene (100 mL, 1 × 2 vol). The filtrate was concentrated under vacuum at 40° C. to 100 mL (2 vol). Charged ethanol (100 mL, 2 vol), and concentrated under vacuum at 40° C. to 100 mL (2 vol). Then charged ethanol (50 mL, 1 vol) and heated mixture to 60° C. to achieve dissolution. Reaction was cooled to 45° C. and pure seeds (0.50 g) were added and crystallization was allowed to set for 1 hour. The resulting mixture was allowed to cool to 5° C. ± 5° C. for 3 hours. Reaction was filtered and isolated solids were washed with prechilled ethanol (15.0 mL, 0.3 vol). The solid was dried in a vacuum oven at 45° C. ± 5° C. with an N₂ bleed for 16 hours to afford a yellow solid (12.27 g, 43.2% yield). ¹H NMR (300 MHz, CDCl3) δ 8.59-8.58 (dd, 1H), 7.92-7.91 (d, 1H).

### Example B-2: Preparation of 3,5-Dichloropicolinonitrile (DMAc-IPAc Condition)

DMAP salt for step 1 (50.0 g, 1.0eq) was charged to a 1-L 2 neck RBF fitted with a condenser, mechanical stirrer and oil bath. IPAc (289.5 g, 6.5 vol) was charged followed by DMAc (16.5 g, 0.5 vol). The reaction was agitated at room temperature. Sodium cyanide (12.06 g, 24.6 mmol, 1.5eq) was charged. The reaction was heated to 90° C. ± 5° C. with stirring. The reaction was monitor by GC analysis for % conversion (highest 82.11% at 5 hours). The reaction was cooled to 50° C. at 5.5 hours filtered and washed with IPAc (2 × 50 mL, 2 × 1 vol). The filtrate was concentrated under vacuum to a residual oil. Charged heptane (350 mL, 7 vol), water (150 mL, 3 vol) and concentrated HCl (14.6 mL, 1 eq (based on DMAP)) and agitated mixture. Charged 10% celite (5.0 g) and heated mixture to 90° C. for 1 hour. Cooled reaction to 70° C. and filtered then washed filter cake with heptane (100 mL, 1 x 2 vol). Separated resulting filtrate layers and retained organic phase. Charged 10% activated carbon (5.0 g) and heated to 90° C. with agitation for 30 minutes. Reaction wash filtered through celite bed generated in last filtration and the resulting bed was wash with heptane (50 mL, 1 × 1 vol). The filtrate was concentrated to half the volume under vacuum and then cooled to 0° C. ± 5° C. and allowed to agitate for 15 minutes. Solids formed and were filtered and washed with heptane (25 mL, 1 × 0.5 vol). The solid was dried under vacuum to produce a yellow solid with purity by AUC HPLC 97%. The resulting solids were suspended in ethanol (60 mL, 4 vol) and heated to 60° C. for 30 minutes to ensure complete dissolution. Reaction was cooled to 45° C. and pure seeds were added and crystallization was allowed to set for 1 hour. The resulting mixture was allowed to cool to 0° C. 5° C. for 15 minutes. Reaction was filtered and isolated solids were washed with prechilled ethanol (15.0 mL, 1.0 vol). The solid was dried in a vacuum oven at 30° C. with an N₂ bleed to afford an off-white solid (14.28 g, 50.28% yield). ¹H NMR (300 MHz, CDCl3) δ 8.59-8.58 (dd, 1H), 7.92-7.91 (d, 1H).

### Example B-3: Preparation of 3,5-Dichloropicolinonitrile (Formula (I-F) Route)

Preparation of 2-fluoro-3,5-dichloropyridine.

2,3,5-Trichloropyridine (TCP, 50 g, 274.077 mmol, 1.0 eq), was taken in a roundbottom flask under N₂ atmosphere, spray dried KF (24.0 g, 413.08 mmol, 1.5 eq), TMAC (3.0 g, 27.407 mmol, 0.1 eq), and NMP (154.5 g, 150 mL, 3 vol) were added at room temperature under N₂ atmosphere. The reaction was heated at about 150° C. (inside temperate) for 4 h. The reaction was then cooled to room temperature and filtered. The resulting solids (filter cake) were washed with MTBE (100 ml, 2 vol). The filtrate was combined and used in next step.

Preparation of 3,5-dichloropicolinonitrile.

To the filtrate from last step (244.3 g) was added water (8 mL, 3%) and NaCN (ball milled powder, 20 g, 408.16 mmol, 1.5 eq) at room temperature under N₂ atmosphere. The reaction was heated at about 60° C. (inside temperate) for 4 h and then cooled to room temperature. Water (300 mL, 6 vol) and MTBE (250 mL, 5 vol) were added to the reaction mixture and stirred for 1 h at room temperature. The organic (MTBE) layer was separated and washed with water (150 mL, 2 times). The mixture (organic layer) was then concentrated by simple distillation (about 90% of MTBE was removed). Heptane (270 ml, 10 vol) was added to the residue at about 50° C., and the resulting mixture was heated at reflux to remove the remaining MTBE. Then, the mixture was cooled to about 70° C., and charcoal (10 %wt, 3 g) was added. The mixture was allowed to reflux for 30 min and then cooled to about 80° C., followed by filtering through a celite pad (the filter funnel was heated during the filtration). The filtrate was cooled to about 0° C. and filtered, the resulting solids (filter cake) were washed with cold heptane (50 ml). The solids were dried at room temperature (flowing N₂) to afford about 18 g off-white solid (40% yield).

Recrystallization in EtOH: the resulting solids from last step were suspended in ethanol (90 mL, 5 vol) and heated to 75° C. for 1 h before slowly cooling down to 0° C. The mixture was filtered and the isolated solids were washed with cold EtOH (1 vol, 18 mL) to afford 16 g product (crop-1, 35% yield). The filtrate was concentrated to afford 1.2 g product (crop-2, 2.6% yield).

### Example C: Preparation of a Compound of Formula (IV)

### Example C-1: Preparation of 5-(3-chlorophenyl)-3-chloro-2-cyanopyridine

A 20 L reactor equipped with a mechanical stirrer, dip tube, thermometer and nitrogen inlet was charged with (3-chlorophenyl)boronic acid (550 g, 3.52 mol), 3,5-dichloro-2-cyanopyridine (639 g, 3.69 mol), K₂CO₃ (5.5 g, 40 mmol), [1,1′-bis(diphenyphosphino)ferrocene]dichloro-palladium(II) [PdCl2(dppf)] (11.5 g, 140 mmol), and dimethylformamide (3894 g, 4.125 L). The reaction solution was agitated and purged with nitrogen through the dip-tube for 30 minutes. Degassed water (413 g) was then charged to the reaction mixture while maintaining a temperature of less than 50° C. 25 hours. The reaction was determined to be complete due to the disappearance of 3,5-dichloro-2-cyanopyridine as measured by TLC analysis using ethyl acetate/methanol (4:1) as the mobile phase and UV 435 nm to visualize the reaction components. The reaction solution was then cooled to 5° C. and charged with heptane (940 g, 1.375 L) and agitated for 30 minutes. Water (5.5 L) was charged and the mixture was further agitated for 1 hour as the temperature was allowed to rise to 15° C. The solid product was isolated by filtration and washed with water (5.5 L) followed by heptane (18881 g, 2750 ML). The resulting cake was air dried under vacuum for 18 hours and then triturated with a mixture of 2-propanol (6908 g, 8800 mL and heptane (1 g, 2200 mL at 50° C. for 4 hours, cooled to ambient temperature and then agitated at ambient temperature for 1 hour. The product was then isolated by filtration and washed with cold 2-propanol (3450 g, 4395 mL) followed by heptane (3010 g, 4400 mL). The resulting solid was dried under high vacuum at 40° C. for 64 hours to afford 565.9 g (65% yield) of the desired product as a beige solid. Purity by HPLC was 98.3%. ¹H NMR (DMSO-d₆) δ 9.12 (d, 1H), 8.70 (d, 1H), 8.03 (t, 01H) 7.88 (m, 1H), and 7.58 (m, 2H).

### Example C-2: Preparation of 5-(3-chlorophenyl)-3-methoxy-2-cyanopyridine

A 20 L reactor equipped with a mechanical stirred, condenser, thermometer and nitrogen inlet was charged with 5-(3-chlorophenyl)-3-chloro-2-cyanopyridine, 1, (558 g, 2.24 mol) and methanol as needed, followed by sodium methoxide (25% solution in methanol, 726.0 g, 3.36 mol). With agitation, the reaction solution was heated to reflux for 24 hours, resulting in a beige-colored suspension. The reaction was determined to be complete due to the disappearance of 5-(3-chlorophenyl)-3-chloro-2-cyanopyridine as measured by TLC analysis using hexane/ethyl acetate (6:3) as the mobile phase and UV 435 nm to visualize the reaction components. The reaction mixture was cooled to 5° C. and then charged with water (5580 mL). The resulting slurry was agitated for 3 hours at 5° C. The solid product was isolated by filtration and washed with water (5580 mL) until the filtrate had a pH of 7. The filter cake was air dried under vacuum for 16 hours. The filter cake was then charged back to the reactor and triturated in MeOH (2210 g, 2794 mL) for 1 hour at ambient temperature. The solid was collected by filtration and washed with MeOH (882 g, 1116 mL, 5° C.) followed by heptane (205 mL, 300 mL), and dried under high vacuum at 45° C. for 72 hours to afford 448 g (82% yield) of the desired product as an off-white solid. Purity by HPLC was 97.9%. ¹H NMR (DMSO-d₆) δ 8.68 (d, 1H), 8.05 (d, 1H), 8.01 (s, 1H) 7.86 (m, 1H), 7.59 (s, 1H), 7.57 (s, 1H) and 4.09 (s, 3H).

### Example D: Preparation of 5-(3-chlorophenyl)-3-hydroxypyridine-2-carboxylic Acid

### Example D-1: Preparation of 5-(3-chlorophenyl)-3-hydroxypyridine-2-carboxylic Acid - Route 1

A 20 L reactor equipped with a mechanical stirrer, condenser, thermometer, nitrogen inlet and 25% aqueous NaOH trap was charged 5-(3-chlorophenyl)-3-methoxy-2-cyanopyridine, 2, (440.6 g, 1.8 mol) and 37% aqueous solution of HCl (5302 g). While being agitated, the reaction solution was heated to 102° C. for 24 hours. Additional 37% aqueous HCl (2653 g) was added followed by agitation for 18 hours at 104° C. The reaction contents was then cooled to 5° C., charged with water (4410 g) and then agitated at 0° C. for 16 hours. The resulting precipitated product was isolated by filtration and washed with water until the filtrate had a pH of 6 (about 8,000 L of water). The filter cake was pulled dry under reduced pressure for 2 hours. The cake was then transferred back into the reactor and triturated in THF (1958 g, 2201 mL) at ambient temperature for 2 hours. The solid product was then isolated by filtration and washed with THF (778 g, 875 mL) and dried under reduced pressure at 5° C. for 48 hours to afford 385 g (89% yield) of the desired product as an off-white solid. HPLC purity was 96.2%. ¹H NMR (DMSO-d₆) δ 8.52 (d, 1H), 7.99 (d, 1H), 7.95 (s, 1H) 7.81 (t, 1H), 7.57 (s, 1H), and 7.55 (s, 1H).

### Example D-2: Preparation of 5-(3-chlorophenyl)-3-hydroxypyridine-2-carboxylic Acid - Route 2

To a 50 mL round bottom flask adapted for magnetic stirring and fitted with a reflux condenser was charged 5-(3-chlorophenyl)-3-methoxy-2-cyanopyridine, 2, (1 g, 4 mmol) and a 48% aqueous solution of HBr (10 mL). While being stirred, the reaction solution was heated to reflux for 20 hours. The reaction was determined to be complete due to the disappearance of 5-(3-chlorophenyl)-3-methoxy-2-cyanopyridine as measured by TLC analysis using hexane/ethyl acetate (6:3) as the mobile phase and UV 435 nm to visualize the reaction components. The reaction contents was then cooled to 0° C. to 5° C. with stirring and the pH was adjusted to approximately 2 by the slow addition of 50% aqueous NaOH. Stirring was then continued at 0° C. to 5° C. for 3 hours. The resulting solid was collected by filtration and washed with water, then hexane. The resulting cake was dried in vacuo at 40° C. to afford 1.03 g (quantitative yield) of the desired product as an off-white solid. ¹H NMR (DMSO-d₆) δ 8.52 (d, 1H), 7.99 (d, 1H), 7.95 (s, 1H) 7.81 (t, 1H), 7.57 (s, 1H), and 7.55 (s, 1H).

### Example E: Preparation of Vadadustat

### Example E-1: Preparation of Vadadustat - Route 1

Preparation of 5-(3-chlorophenyl)-2-(N-glycine methylester carboxylic amide)-3-(2,2-dimethyl-1-oxopropoxy) pyridine (Method 1): 3-Hydroxy 5-(3-chlorophenyl)- 2-carboxy-pyridine (1.00 wt) and tetrahydrofuran (4.48 wt/wt) was charged to a reactor, followed by N,N-diisopropyethylamine (1.21 wt/wt). Pivaloyl chloride (1.05 wt/wt) was added at about 0° C. and the mixture was agitated until the reaction was deemed to be completed. Tetrahydrofuran (2.59 wt/wt) and glycine methyl ester hydrochloride (0.64 wt/wt) were added at about 0° C. and N,N-diisopropyethylamine (0.78 wt/wt) was added at about 0° C. The mixture was agitated at about 0° C. and at ambient temperature until the reaction was deemed completed. The reaction solvent tetrahydrofuran was exchanged for ethanol at elevated temperature under vacuum. Water (8.00 wt/wt) was added at about 40° C. The resulting suspension was agitated at ambient temperature, filtered and washed with ethanol and water.

Preparation of 5-(3-chlorophenyl)-2-(N-glycine methylester carboxylic amide)-3-(2,2-dimethyl-1-oxopropoxy) pyridine (Method 2): 3-Hydroxy 5-(3-chlorophenyl)- 2-carboxy-pyridine (1.00 wt) and tetrahydrofuran (4.48 wt/wt) was charged to a reactor, followed by N,N-diisopropyethylamine (1.21 wt/wt). Pivaloyl chloride (1.05 wt/wt) was added at about 0° C. and the mixture was agitated until the reaction was deemed to be completed. Tetrahydrofuran (2.59 wt/wt) and glycine methyl ester hydrochloride (0.64 wt/wt) were added at about 0° C. and N,N-diisopropyethylamine (0.78 wt/wt) was added at about 0° C. The mixture was agitated at about 0° C. and at ambient temperature until the reaction was deemed completed. The reaction solvent tetrahydrofuran was exchanged for ethanol at elevated temperature under vacuum. Water (8.00 wt/wt) was added at about 40° C., followed by an additional amount of N,N-diisopropyethylamine (0.077 wt/wt). The suspension was agitated at ambient temperature, filtered and washed with ethanol and water. ¹H NMR (300 MHz, DMSO-d₆) δ 9.064 (t, j = 6.1 Hz, 1H), 8.947 (d, j = 2.0 Hz, 1H), 8.161 (d, j = 2.0 Hz, 1H), 7.999 (m, 1H), 7.870 (m, 1H), 7.568 (m, 2H), 4.024 (d, j = 6.1 Hz, 2H), 3.656 (s, 3H), 1.332 (s, 9H).

Preparation of vadadustat: 5-(3-chlorophenyl)- 2-(N-glycine methylester carboxylic amide)- 3-(2,2-dimethyl-1-oxopropoxy) pyridine, 2-methyl-tetrahydrofuran (6.92 wt/wt) and water (3.24 wt/wt) were charged into a reactor. A potassium hydroxide solution of approximately 45% (1.50 wt/wt) was added and the mixture agitated at ambient temperature until the reaction was deemed to be completed. Water (3.73 wt/wt) was charged and the mixture was acidified with concentrated aqueous HCl (about 1.3 wt/wt) at ambient temperature. The lower aqueous phase was discharged, and water was added to the organic extract at about 45° C. The lower aqueous phase was discharged and the organic phase was polish filtered. 2-Methyl-tetrahydrofuran (8.30 wt/wt) was charged and the mixture concentrated at about 45° C. under vacuum to about 5 volumes. n-Heptane (0.99 wt/wt) was and 5-(3-chlorophenyl)-2-(N-glycine carboxylic amide)-3-hydroxypyridine seeds (0.005 wt/wt) were added at about 45° C. n-Heptane (5.62 wt/wt) was charged in about 2 h and the suspension was agitated for about 1 h at about 45° C. The suspension was concentrated to about 6.5 volumes at elevated temperature under vacuum, followed by agitation at about 75° C. The suspension was cooled to ambient temperature, agitated and filtered. The wet cake was washed with n-heptane (3.31 wt/wt) and dried at about 50° C. und vacuum to yield white to beige crystals in about 90% yield and a purity of about 100% by HPLC from the charged amount of 3-hydroxy 5-(3-chlorophenyl)- 2-carboxy-pyridine (Compound 5). ¹H NMR (DMSO-d₆) δ 12.84 (s, 1H), 12.39 (s, 1H), 9.39 (t, 1H), 8.56 (d, 1H), 7.94 (s, 1H), 7.81 (m, 2H), 7.55 (q, 2H), and 4.02 (d, 2H).

### Example E-2: Preparation of Vadadustat - Route 2

Preparation of methyl {[5-(3-chlorophenyl)-3-hydroxypyridin-2-yl]amino}acetate: To a 50 mL round bottom flask adapted for magnetic stirring and fitted with a nitrogen inlet tube was charged 5-(3-chlorophenyl)-3-hydroxypyridine-2-carboxylic acid, 3, (1 gm, 4 mmol), N,N′-carbonyldiimidazole (CDI) (0.97 g, 6 mmol) and dimethyl sulfoxide (5 mL). The reaction mixture was stirred at 45° C. for about 1 hour then cooled to room temperature. Glycine methyl ester hydrochloride (1.15 g, 12 mmol) is added followed by the dropwise addition of diisopropylethylamine (3.2 mL, 19 mmol). The mixture was then stirred for 2.5 hours at room temperature after which water (70 mL) was added. The contents of the reaction flask was cooled to 0° C. to 5° C. and 1N HCl was added until the solution pH is approximately 2. The solution was extracted with dichloromethane (100 mL) and the organic layer was dried over MgSO₄ for 16 hours. Silica gel (3 g) is added and the solution slurried for 2 hours after which the solids are removed by filtration. The filtrate is concentrated to dryness under reduced pressure and the resulting residue was slurried in methanol (10 mL) for two hours. The resulting solid was collected by filtration and washed with cold methanol (20 mL) then hexane and the resulting cake is dried to afford 0.85 g of the desired product as an off-white solid. The filtrate was treated to afford 0.026 g of the desired product as a second crop. The combined crops afford 0.88 g (68% yield) of the desired product. ¹H NMR (DMSO-d₆) δ 12.3 (s, 1H), 9.52 (t, 1H), 8.56 (d, 1H), 7.93 (s, 1H), 7.80 (q, 2H), 7.55 (t, 2H), 4.12 (d, 2H), and 3.69 (s, 3H).

Preparation of vadadustat: To a 50 mL flask is charged methyl {[5-(3-chlorophenyl)-3-hydroxypyridin-2-yl]amino}-acetate, 4, (0.45 g, 1.4 mmol), tetrahydrofuran (4.5 mL) and 1 M NaOH (4.5 mL, 4.5 mmol). The mixture was stirred for 2 hours at room temperature after which it was determined by TLC analysis using hexane/ethyl acetate (6:3) as the mobile phase and UV 435 nm to visualize the reaction components that the reaction was complete. The reaction solution was adjusted to pH 1 with concentrated HCl and the solution was heated at 35° C. under vacuum until all of the tetrahydrofuran had been removed. A slurry forms as the solution is concentrated. With efficient stirring the pH is adjusted to ~2 with the slow addition of 1 M NaOH. The solid which forms was collected by filtration, washed with water, followed by hexane, then dried under vacuum to afford 0.38 g (88% yield) of the desired product as a white solid. ¹H NMR (DMSO-d₆) δ 12.84 (s, 1H), 12.39 (s, 1H), 9.39 (t, 1H), 8.56 (d, 1H), 7.94 (s, 1H), 7.81 (m, 2H), 7.55 (q, 2H), and 4.02 (d, 2H).

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A process for preparing a compound of Formula (IV) 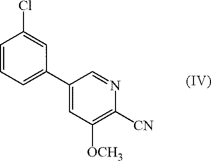 [SMILES: COc1cc(-c2cccc(Cl)c2)cnc1C#N]
or a pharmaceutically acceptable salt thereof, comprising:
(a) contacting a compound of Formula (I), 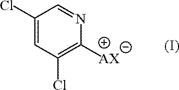
or a tautomer thereof, with a cyanide source to obtain a compound of Formula (II), 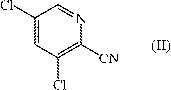
or a salt thereof, wherein A is an activator and X is a halogen; and
(b) contacting the compound of Formula (II) or a salt thereof with a compound of Formula (III) 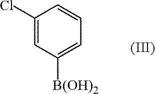
or a salt thereof, to form a compound of Formula (IVa), 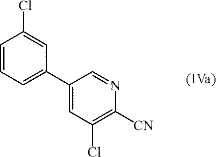 [SMILES: N#Cc1ncc(-c2cccc(Cl)c2)cc1Cl]
or a pharmaceutically acceptable salt thereof.

2. A process for preparing a compound of Formula (IV) 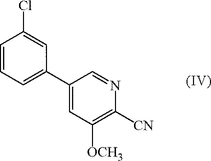 [SMILES: COc1cc(-c2cccc(Cl)c2)cnc1C#N]
or a pharmaceutically acceptable salt thereof, comprising:
(a-F) contacting a compound of Formula (I-F), 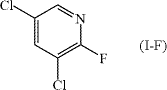
with a cyanide source to obtain a compound of Formula (II), 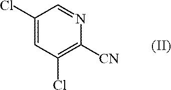
or a salt thereof; and
(b) contacting the compound of Formula (II) or a salt thereof with a compound of Formula (III) 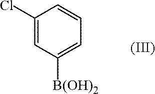
or a salt thereof, to form a compound of Formula (IVa), 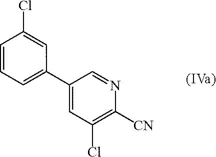 [SMILES: N#Cc1ncc(-c2cccc(Cl)c2)cc1Cl]
or a pharmaceutically acceptable salt thereof.

3. The process of claim 1 or 2, further comprising a step that is
(c) contacting the compound of Formula (IVa), or a salt thereof, with methanol or an inorganic salt thereof to form a compound of Formula (IV), 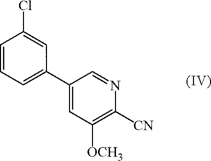 [SMILES: COc1cc(-c2cccc(Cl)c2)cnc1C#N]
or a pharmaceutically acceptable salt thereof.

4. A process for preparing a compound of Formula (V) 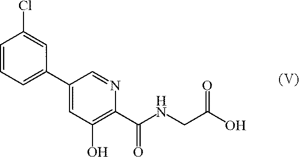 [SMILES: O=C(O)CNC(=O)c1ncc(-c2cccc(Cl)c2)cc1O]
or a pharmaceutically acceptable salt thereof comprising contacting a compound of Formula (IV) with glycine methyl ester hydrochloride, wherein the compound of Formula (IV) is obtained by the process of
claim 1 or claim 2.

5. The process of any one of claims 1, 3, and 4, wherein A is a nucleophilic amine, a triarylphosphine, or a trialkylphosphine.

6. The process of any one of claims 1 and 3-5, wherein A is DMAP (4-dimethylaminopyridine), DABCO (1,4-diazabicyclo[2.2.2]octane), DBU (1,8-Diazabicyclo[5.4.0]undec-7-ene), DBN (1,5-Diazabicyclo[4.3.0]non-5-ene), triphenylphosphine, trimethylphosphine, or tributylphosphine.

7. The process of claim 6, wherein A is DMAP.

8. The process of any one of claims 1 and 3-7, wherein X is F, Cl or Br.

9. The process of claim 8, wherein the compound of Formula (I) has the following structure, 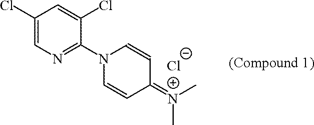
or a tautomer thereof.

10. The process of any one of claims 1-9, wherein the cyanide source is an inorganic cyanide salt or an organosilicon cyanide compound.

11. The process of any one of claims 1-10, wherein the cyanide source is NaCN, KCN, TMSCN (trimethylsilyl cyanide), Zn(CN)₂, or CuCN.

12. The process of claim 11, wherein the cyanide source is NaCN.

13. The process of any one of claims 1-12, wherein step (a) or step (a-F) occurs in the presence of a solvent.

14. The process claim 13, wherein the solvent is an ester solvent, an aprotic solvent, or a combination thereof.

15. The process of claim 14, wherein the ester solvent is isopropyl acetate (IPAc), ethyl propionate, or ethyl acetate.

16. The process of claim 14, wherein the aprotic solvent is methyl tert-butyl ether (MTBE), N-methyl-2-pyrrolidone (NMP), acetonitrile (ACN), acetone, heptane, methylene chloride (MC), tetrahydrofuran (THF), dimethylformamide (DMF), dimethyl sulfoxide (DMSO), or dimethyl acetamide (DMAc).

17. The process of claim 16, wherein the aprotic solvent is tetrahydrofuran (THF), dimethylformamide (DMF), dimethyl sulfoxide (DMSO), or dimethyl acetamide (DMAc).

18. The process of claim 16, wherein the aprotic solvent is methyl tert-butyl ether (MTBE), N-methyl-2-pyrrolidone (NMP), acetonitrile (ACN), acetone, heptane, or methylene chloride (MC).

19. The process of claim 13, wherein step (a) occurs in the presence of a solvent that is IPAc.

20. The process of claim 13, wherein step (a) occurs in the presence of a solvent that is a combination comprising IPAc and DMAc.

21. The process of claim 20, wherein IPAc and DMAc are present in a ratio ranging from about 19:1 to 1:1 IPAc:DMAc.

22. The process of any one of claims 15-21, wherein step (a) occurs at a temperature of about 80-100° C. or about 85-95° C.

23. The process of claim 22, wherein step (a) occurs at a temperature of about 90° C.

24. The process of claim 13, wherein step (a-F) occurs in the presence of a solvent that is NMP.

25. The process of claim 13, wherein step (a-F) occurs in the presence of a solvent that is a combination comprising NMP and MTBE.

26. The process of claim 25, wherein NMP and MTBE are present in a ratio ranging from about 1:3 to 6:1 NMP:MTBE.

27. The process of claim 26, wherein NMP and MTBE are present in a ratio of about 3:2 NMP:MTBE.

28. The process any one of claims 24-27, wherein the solvent comprises about 1% to 5% water.

29. The process of claim 28, wherein the solvent comprises about 3% water.

30. The process of any one of claims 13-18 and 24-29, wherein step (a-F) occurs at a temperature of about 25-100° C.

31. The process of claim 30, wherein step (a-F) occurs at a temperature of about 60° C. .

32. The process of any one of claims 1 and 3-23, wherein the compound of Formula (I) is prepared by a process comprising: (i) contacting a compound of Formula (VI) or a salt thereof, 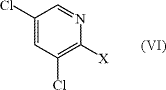
with the activator, wherein X is a halogen.

33. The process of claim 32, wherein the compound of Formula (VI) is 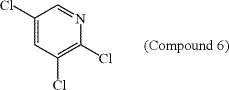
.

34. The process of claim 32 or 33, wherein step (i) further comprises a solvent.

35. The process of claim 32 or 33, wherein step (i) occurs in a neat condition.

36. The process of claim 35, wherein step (i) comprises contacting about 3-10 equivalents of the compound of Formula (VI) with about 1 equivalent of the activator to obtain about 1 equivalent of the compound of Formula (I) and an amount of the compound of Formula (VI).

37. The process of claim 35, wherein step (i) comprises contacting about 3 equivalents of the compound of Formula (VI).

38. The process of any one of claims 32-37, wherein the process further comprises:
(ii) isolating the compound of Formula (I) obtained in step (i) from the amount of the compound of Formula (VI).

39. The process of claim 38, wherein the process further comprises:
(iii) optionally adding about 1 additional equivalent of the compound of Formula (VI) to the remaining amount of the compound of Formula (VI) from step (ii);
(iv) repeating step (i) using the compound of Formula (VI) in step (ii) or step (iii) and about 1 equivalent of the activator; and
(v) optionally repeating steps (ii) to (iv).

40. The process of claim 39, wherein step (iii) comprises in total at least about 3 equivalents of the compound of Formula (VI).

41. The process of claim 39 or 40, wherein the process comprises about 2-8 total cycles to form the compound of Formula (I).

42. The process of any one of claims 2-4, 10-18, and 24-31, wherein the compound of Formula (I-F) is prepared by a process comprising a step (i-F), wherein said step comprises contacting a compound of Formula (VI) or a salt thereof 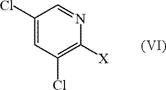
with a fluoride source, and wherein X is a halogen.

43. The process of claim 42, wherein the compound of Formula (VI) is 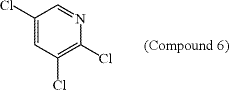
.

44. The process of claim 41 or 42, wherein the fluoride source is KF.

45. The process of claim 43, wherein the KF is spray-dried KF.

46. The process of any one of claims 42-45, wherein step (i-F) further comprises a solvent.

47. The process of claim 46, wherein the solvent is DMF, DMSO, DMF, DMAc, sulfolane, NMP, MTBE, acetonitrile (ACN), butyronitrile, or a combination thereof.

48. The process of claim 47, wherein the solvent is NMP.

49. The process of any one of claims 42-48, wherein step (i-F) further comprises a phase-transfer catalyst.

50. The process of claim 49, wherein the phase-transfer catalyst is tetramethylammonium chloride (TMAC), tetramethylammonium fluoride (TMAF), tetrabutylammonium fluoride (TBAF), tetrabutylammonium chloride (TBAC), benzyltriethylammonium chloride (BTEAC), phenyltrimethylammonium chloride (PhTMAC), DMAP-BnCl (salt made from 4-dimethylaminopyridine and benzyl chloride), 18-Crown-6, or a combination thereof.

51. The process of claim 50, wherein the phase-transfer catalyst is TMAC.

52. The process of any one of claims 42-51, wherein step (i-F) occurs at a temperature of above about 100° C., of about 110 to about 150° C., of about 130 to about 170° C., or of about 140 to about 160° C.

53. The process of claim 45, wherein step (i-F) occurs at a temperature of about 150° C.

54. The process of any one of claims 42-52, wherein step (i-F) comprises contacting about 1 equivalent of the compound of Formula (VI) with about 1-3 equivalents of the fluoride source.

55. The process of claim 54, wherein step (i-F) comprises contacting about 1 equivalent of the compound of Formula (VI) with about 1.5 equivalents of the fluoride source.

56. The process of any one of claims 42-55, wherein step (i-F) comprises contacting about 1 equivalent of the compound of Formula (VI) with about 0.01-0.5 equivalent of the phase-transfer catalyst.

57. The process of any one of claims 56, wherein step (i-F) comprises contacting about 1 equivalent of the compound of Formula (VI) with about 0.1 equivalent of the phase-transfer catalyst.

58. The process of any one of claims 42-57, wherein step (i-F) further comprises:
(ii-F) filtering the reaction mixture obtained from step (i-F); and/or
(iii-F) optionally isolating the compound of Formula (I-F) obtained in step (i-F) or (ii-F) from the amount of the compound of Formula (VI).

59. A compound of Formula (I) 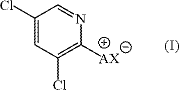
or a tautomer thereof, wherein:
A is an activator; and
X is a halogen.

60. The compound of claim 59, wherein the activator is DMAP, DABCO, DBU, DBN, triphenylphosphine, tributylphosphine, or trimethylphosphine.

61. The compound of claim 59 or 60, wherein X is F, Cl or Br.

62. The compound of any one of claims 59-61, wherein the compound is 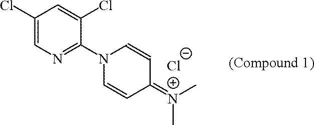
or a tautomer thereof.

63. The compound of any one of claims 59-62, isolated in solid form.
